(19) United States
(12) Patent Application Publication
(10) Pub. No.: US 2025/0213115 A1
(43) Pub. Date: Jul. 3, 2025

(54) IMPLANTABLE MEDICAL DEVICE WITH SENSING AND COMMUNICATION FUNCTIONALITY UTILIZING A SUBSTRATE ANTENNA

(71) Applicant: CANARY MEDICAL SWITZERLAND AG, Baar (CH)

(72) Inventor: Kimberly Salant, Asbury, NJ (US)

(21) Appl. No.: 18/846,520

(22) PCT Filed: Mar. 14, 2023
(86) PCT No.: PCT/US2023/015224

§ 371 Date: Sep. 12, 2024

Related U.S. Application Data
(60) Provisional application No. 63/319,717, filed on Mar. 14, 2022.

(51) Int. Cl. A61B 5/00; A61B 5/11; A61B 17/70; H01Q 1/27; H01Q 7/00

(52) U.S. Cl. CPC A61B 5/0031; A61B 5/1121; A61B 5/686; A61B 17/70; H01Q 1/273; H01Q 7/00; A61B 2562/0219

(57) ABSTRACT

An implantable system for a body part of a patient can include an electronic circuitry. The electronic circuitry can include at least one sensor, a processor configured to receive data obtained by the at least one sensor, a communication circuitry connected to the processor and configured to transmit the data obtained by the at least one sensor, and an antenna comprising a plurality of conductive traces present on a biocompatible substrate, the antenna connected to the communication circuitry and further configured to facilitate transmission of the data. The body part can be a joint, such as the hip joint, or the spine.

### CROSS-REFERENCE TO RELATED APPLICATIONS

Any and all applications for which a foreign or domestic priority claim is identified in the Application Data Sheet as filed with the present application are hereby incorporated by reference.

### TECHNICAL FIELD

The present disclosure relates generally to intelligent implants, and more particularly, to implantable medical devices with implantable reporting processors that sample, record, and transmit information related to the placement and integrity of an implanted device, and the health of the patient in which the device is implanted.

### BACKGROUND

After treating an internal injury or other internal defect, it can be difficult to monitor the progress of the patient's recovery. For example, after a knee arthroplasty (such as, total knee arthroplasty (TKA)), hip replacement, or shoulder replacement has been performed and the patient begins to move with the implant, problems may occur and are sometimes hard to identify. Clinical exams are often limited in their ability to detect failure of the prosthesis; therefore, additional monitoring is often required such as CT scans, MRI scans or even nuclear scans. Given the continuum of care requirements over the lifetime of the prosthesis, patients are encouraged to visit their clinician periodically (such as, annually) to review their health condition, monitor other joints, and assess the implant's function. Such assessments are often subjective and lack temporal resolution to delineate small changes in functionality that could be a pre-cursor to larger mobility issues. The long-term (>1 year) follow up of patients also poses a problem in that patients do not consistently see their clinicians annually. Rather, they often seek additional consultation only when there is pain or other symptoms.

Currently, there is no mechanism for reliably detecting misplacement, instability, or misalignment of the implant without clinical visits and the hands and visual observations of an experienced health care provider. Even then, early identification of subclinical problems or conditions is either difficult or impossible since they are often too subtle to be detected on physical exam or demonstratable by radiographic studies. Furthermore, if detection were possible, corrective actions would be hampered by the fact that the specific amount of movement and/or degree of improper alignment cannot be accurately measured or quantified, making targeted, successful intervention unlikely. Existing external monitoring devices do not provide the fidelity required to detect instability since these devices are separated from the implant by skin, muscle, and fat—each of which masks the mechanical signatures of instability and introduce anomalies such as flexure, tissue-borne acoustic noise, inconsistent sensor placement on the surface, and inconsistent location of the external sensor relative to the implant.

In general, correct placement of a medical implant can be challenging to the surgeon and various complications may arise during insertion of any medical implant (whether it is an open surgical procedure or a minimally invasive procedure). For example, a surgeon may wish to confirm correct anatomical alignment and placement of the implant within surrounding tissues and structures. This can however be difficult to do during the procedure itself, making intraoperative corrective adjustments difficult.

In addition, a patient may experience a number of complications post-procedure. Such complications include neurological symptoms, pain, malfunction (blockage, loosening, etc.) and/or wear of the implant, movement or breakage of the implant, inflammation and/or infection. While some of these problems can be addressed with pharmaceutical products and/or further surgery, they are difficult to predict and prevent; often early identification of complications and side effects, although desirable, is difficult or impossible.

The present disclosure is directed to identifying, locating and/or quantifying these problems, particularly at an early stage, and providing methods and devices to remedy these problems.

All of the subject matter discussed in the Background section is not necessarily prior art and should not be assumed to be prior art merely as a result of its discussion in the Background section. Along these lines, any recognition of problems in the prior art discussed in the Background section or associated with such subject matter should not be treated as prior art unless expressly stated to be prior art. Instead, the discussion of any subject matter in the Background section should be treated as part of the inventor's approach to the particular problem, which in and of itself may also be inventive.

### SUMMARY

This Brief Summary has been provided to introduce certain concepts in a simplified form that are further described in detail below in the Detailed Description. Except where otherwise expressly stated, this Brief Summary is not intended to identify key or essential features of the claimed subject matter, nor is it intended to limit the scope of the claimed subject matter.

Certain aspects of the present disclosure are directed toward an implantable system for a body part of a patient. The system can include an electronic circuitry with at least one sensor, a processor configured to receive data obtained by the at least one sensor, and a communication circuitry connected to the processor and configured to transmit the data obtained by the at least one sensor. The system can include an antenna comprising a plurality of conductive traces positioned (such as, printed, painted, or otherwise deposited) on a substrate, which can be biocompatible. The antenna can be connected to the communication circuitry and facilitate transmission of the data.

### BRIEF DESCRIPTION OF THE DRAWINGS

Example features of the present disclosure, its nature and various advantages will be apparent from the accompanying drawings and the following detailed description of various embodiments. Non-limiting and non-exhaustive embodiments are described with reference to the accompanying drawings, wherein like labels or reference numbers refer to like parts throughout the various views unless otherwise specified. The sizes and relative positions of elements in the drawings are not necessarily drawn to scale. For example, the shapes of various elements are selected, enlarged, and positioned to improve drawing legibility. The particular shapes of the elements as drawn have been selected for ease of recognition in the drawings. One or more embodiments are described hereinafter with reference to the accompanying drawings in which:

FIG. 1 illustrates an example hip implant, according to certain aspects of the present disclosure.

FIG. 2 illustrates an example sensor environment, according to certain aspects of the present disclosure.

FIG. 3 illustrates an example sensor system for a hip implant, according to certain aspects of the present disclosure.

FIG. 4 illustrates a flow chart of design process or an antenna for use with a sensor system, according to certain aspects of the present disclosure.

FIGS. 5A, 5B and 5C illustrates a loop antenna, substrate antenna, and helix antenna, according to certain aspects of the present disclosure.

FIG. 6 illustrates a cross section view of a sensor assembly with a loop antenna, according to certain aspects of the present disclosure.

FIGS. 7A, 7B, 7C and 7D illustrate gain, impedance, and return loss of the loop antenna, according to certain aspects of the present disclosure.

FIG. 8 illustrates a planar inverted-F antenna, according to certain aspects of the present disclosure.

FIGS. 9A, 9B, 9C, 9D, 9E, 9F and 9G illustrate gain, impedance, return loss, and a matching network of the planar inverted-F antenna, according to certain aspects of the present disclosure.

FIG. 10 illustrates a substrate antenna, according to certain aspects of the present disclosure.

FIGS. 11A and 11B illustrate impedance and return loss of the layer substrate antenna, according to certain aspects of the present disclosure.

FIG. 12 illustrates variation of stub length of the substrate antenna, according to certain aspects of the present disclosure.

FIG. 13 illustrates variation of spacer thickness of the single layer substrate antenna, according to certain aspects of the present disclosure.

FIG. 14 illustrates a multilayer substrate antenna, according to certain aspects of the present disclosure.

FIG. 15 illustrates grounding of an antenna, according to certain aspects of the present disclosure.

FIG. 16 illustrates various substrate antenna designs, according to certain aspects of the present disclosure.

FIG. 17 illustrates comparison of performance of various antennas, according to certain aspects of the present disclosure.

FIG. 18 illustrates a zig-zag pattern for a substrate antenna, according to certain aspects of the present disclosure.

FIGS. 19A and 19B illustrate an implantable medical device with sensing and communication functionality, according to certain aspects of the present disclosure.

FIGS. 20A, 20B and 20C illustrate a matching network and return loss of the substrate antenna, according to certain aspects of the present disclosure.

FIGS. 21A, 21B, 21C, 21D and 21E illustrate comparison performance of various antennas and implementations of a substrate antenna, according to certain aspects of the present disclosure.

FIGS. 22A and 22B illustrates a substrate antenna with covers, according to certain aspects of the present disclosure.

FIGS. 23A, 23B and 23C illustrate various antenna covers, according to certain aspects of the present disclosure.

FIGS. 24A and 24B illustrate return loss of a substrate antenna with covers, according to certain aspects of the present disclosure.

FIGS. 25A, 25B, 25C, 25D and 25E illustrate communication range of various substrate antennas, according to certain aspects of the present disclosure.

FIG. 26A illustrates a sensor assembly in the form of a spinal cage with an insertable cartridge, wherein the cartridge includes an implantable reporting processor.

FIGS. 26B and 26C illustrate perspective views of the insertable cartridge of FIG. 26A.

FIG. 27A illustrates another sensor assembly in the form of a spinal cage with an insertable cartridge, wherein the cartridge includes an implantable reporting processor.

FIGS. 27B, 27C, and 27D illustrate a cross-sectional view of the insertable cartridge of FIG. 27A.

FIG. 28 illustrates a sensor assembly in the form of a spinal cage with an insertable cartridge, wherein the spinal cage includes a window.

FIG. 29A illustrates a cross-sectional medial-lateral view of the sensor assembly of FIG. 23A.

FIG. 29B illustrates a cross-sectional medial-lateral view of the sensor assembly of FIG. 24A.

FIG. 30A illustrates a front posterior-anterior view of a sensor assembly with a spinal cage with an insertable cartridge, wherein the spinal cage does not include a window.

FIG. 30B illustrates a front posterior-anterior view of a sensor assembly with a spinal cage with an insertable cartridge, wherein the spinal cage includes a window.

FIG. 31 illustrates the sensor assembly of any of FIGS. 26A-26C, 27A-27D, 28, 29A-29B, or 30A-30B implanted in the spine of a patient during spinal fusion.

### DETAILED DESCRIPTION

The present disclosure may be understood more readily by reference to the following detailed description of example configurations of “an intelligent implant,” “a sensor assembly,” or “a sensor system” included herein. The following description, along with the accompanying drawings, sets forth certain specific details in order to provide a thorough understanding of various disclosed embodiments. However, one skilled in the relevant art will recognize that the disclosed embodiments may be practiced in various combinations, without one or more of these specific details, or with other methods, components, devices, materials, etc. In other instances, well-known structures or components that are associated with the environment of the present disclosure, including but not limited to the communication systems and networks, have not been shown or described in order to avoid unnecessarily obscuring descriptions of the embodiments. Additionally, the various embodiments may be methods, systems, media, or devices. Accordingly, the various embodiments may be entirely hardware embodiments, entirely software embodiments, entirely firmware embodiments, or embodiments combining or subcombining software, firmware, and hardware aspects.

Prior to setting forth this disclosure in more detail, it may be helpful to an understanding thereof to provide definitions of certain terms to be used herein. Additional definitions are set forth throughout this disclosure. The terms “include” and “comprise,” as well as derivatives thereof, mean inclusion without limitation. The term “or,” is inclusive, meaning and/or. The phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like. The term “controller” or “processor” means any device, system, or part thereof that controls at least one operation, such a device may be implemented in hardware (e.g., electronic circuitry), firmware, or software, or some combination of at least two of the same. The functionality associated with any particular controller may be centralized or distributed, whether locally or remotely. Other definitions of certain words and phrases may be provided within this patent document. Those of ordinary skill in the art will understand that in many, if not most instances, such definitions apply to prior as well as future uses of such defined words and phrases.

An “intelligent medical device” as used in the present disclosure, is an implantable or implanted medical device that desirably replaces or functionally supplements a subject's natural body part. The intelligent medical device can include one of the disclosed sensor assemblies and/or an anchoring (or anchor) structure. The sensor assembly will comprise or be associated with a controller or processor, also referred to as an implantable reporting processor (“IRP”). In one configuration, the intelligent medical device is an implanted or implantable medical device having a sensor assembly with the IRP arranged to perform the functions as described herein. The sensor assembly may perform one or more of the following example actions in order to characterize the post-implantation status of the intelligent medical device: identifying the intelligent medical device or a portion of the intelligent medical device (e.g., the sensor assembly or by recognizing one or more unique identification codes for the intelligent medical device or a portion of the intelligent medical device); detecting, sensing and/or measuring parameters, which may collectively be referred to as monitoring parameters, in order to collect operational, physiological, kinematic or other data about the intelligent medical device or a portion of the intelligent prosthesis (e.g., the sensor assembly) and such data may optionally be collected as a function of time; storing the collected data within the intelligent medical device or a portion of the intelligent medical device (e.g., the sensor assembly); and communicating the collected data and/or the stored data by a wireless means from the intelligent medical device or a portion of the intelligent medical device (e.g., the sensor assembly) to an external computing device. The external computing device may have or otherwise have access to at least one data storage location such as found on a personal computer, a base station, a computer network, a cloud-based storage system, or another computing device that has access to such storage. Non-limiting and non-exhaustive list of configurations of intelligent medical devices include a housing configured to be implanted in a body part.

“Monitoring data,” as used herein, individually or collectively includes some or all data associated with a particular implantable sensor assembly, and available for communication outside of the particular implantable sensor system. For example, monitoring data may include raw data from one or more sensors of the sensor assembly. Monitoring data may also include processed data from one or more sensors, status data, operational data, control data, fault data, time data, scheduled data, event data, log data, and the like associated with the particular sensor assembly. In some cases, high resolution monitoring data includes monitoring data from one, many, or all of the sensors of the sensor assembly that is collected in higher quantities, resolution, from more sensors, more frequently, or the like.

“Sensor” refers to a device that can be utilized to do one or more of detect, measure and/or monitor one or more different aspects of a body tissue (e.g., anatomy, physiology, metabolism, and/or function) and/or one or more aspects of the smart medical device or the sensor system. Representative examples of sensors suitable for use within the present disclosure include, for example, fluid pressure sensors, fluid volume sensors, contact sensors, position sensors, pulse pressure sensors, blood volume sensors, blood flow sensors, chemistry sensors (e.g., for blood and/or other fluids), metabolic sensors (e.g., for blood and/or other fluids), accelerometers, mechanical stress sensors and temperature sensors. Within certain embodiments the sensor can be a wireless sensor, or, within other embodiments, a sensor connected to a wireless microprocessor. Within further embodiments one or more (including all) of the sensors can have a Unique Sensor Identification number (“USI”) which specifically identifies the sensor. In certain embodiments, the sensor is a device that can be utilized to measure in a quantitative manner, one or more different aspects of a body tissue (anatomy, physiology, metabolism, and/or function) and/or one or more aspects of the implant. In certain embodiments, the sensor is an accelerometer that can be utilized to measure in a quantitative manner, one or more different aspects of a body tissue (e.g., function) and/or one or more aspects of the implant (e.g., alignment in the patient).

“Sensor assembly” may refer to one or more components. For example, the “sensor assembly” may be a single component sensor with processing and wireless transmission on board the sensor. In other examples, the “sensor assembly” may be multiple components with a sensor and other components for performing one or more functions described herein.

In order to further understand the various aspects of the invention provided herein, the following sections are provided below: I. Overview; II. Sensor Assembly; III. Additional Embodiments and Terminology; and VI. Example Embodiments.

### I. Overview

The present disclosure provides intelligent implants (or sensor systems or sensor assemblies), e.g., an implantable medical device with an implantable reporting processor (IRP), that may be utilized to monitor and report the status and/or activities of the implant itself, and the patient in which the intelligent implant is implanted. The intelligent implant can be implanted in any joint of body part of a patient. For example, in one embodiment, the intelligent implant is part of an implant system that replaces a joint of a patient, e.g., a knee, shoulder, or hip joint, and allows the patient to have the same, or nearly the same, mobility as would have been afforded by a healthy joint. When the intelligent implant is included in a component of an implant system that replaces a joint, the intelligent implant can monitor displacement or movement of the component or implant system. Examples of joint replacement implant systems in which the intelligent implants disclosed herein may be incorporated, are described in PCT Publication Nos. WO 2014/144107, WO 2014/209916, WO 2016/044651, WO 2017/165717, and WO 2020/247890, the disclosures of which are incorporated herein in their entirety.

In an embodiment, the implantable medical device is a hip implant system or a component of a hip implant system, and in particular a total hip implant for total hip arthroplasty (or total hip replacement). The intelligent implant included in the total hip implant system can monitor and characterize movement of the hip implant. In general, there are three types of three-dimensional motion that the intelligent implant can detect within and around a joint: core gait (or limb mobility in the case of a shoulder or elbow arthroplasty), macroscopic instability, and microscopic instability. Details of these types of motion are described in detail in PCT Publication Nos. WO 2017/165717 and WO 2020/247890.

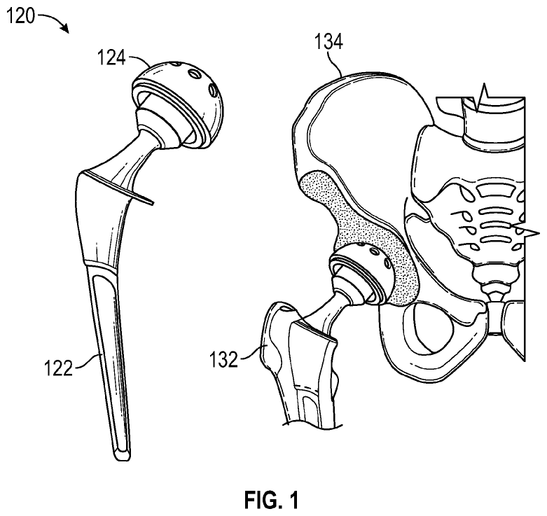

FIG. 1 illustrates a femoral implant 120 (or femoral prosthesis or hip implant) for total hip replacement. The femoral implant 120 can be a cemented implant (which can be fixed into place with bone cement) or cementless implant. The hip joint is a ball-and-socket joint. The socket is formed by the acetabulum, which is part of the pelvis 134. The ball is the femoral head, which is the upper end of the femur 132. The femoral implant 120 can include a femoral stem 122 and a femoral head 124. During total hip replacement, the damaged femoral head can be removed and replaced with a femoral stem 122 that is placed into the femur 132. A ball-shaped component (femoral head 124) can be placed on the upper part of the femoral stem 122. This component replaces the damaged femoral head that was removed.

In some cases, the implantable medical device is a knee implant system or a component of a knee implant system, and in particular a total knee implant for total knee arthroscopy. The intelligent implant included in the total knee implant system can monitor and characterize movement of the knee implant. In some instances, the implantable medical device can be a shoulder implant, an elbow implant, or an implant for another joint or body part. More generally, the implantable medical device can be any implantable device.

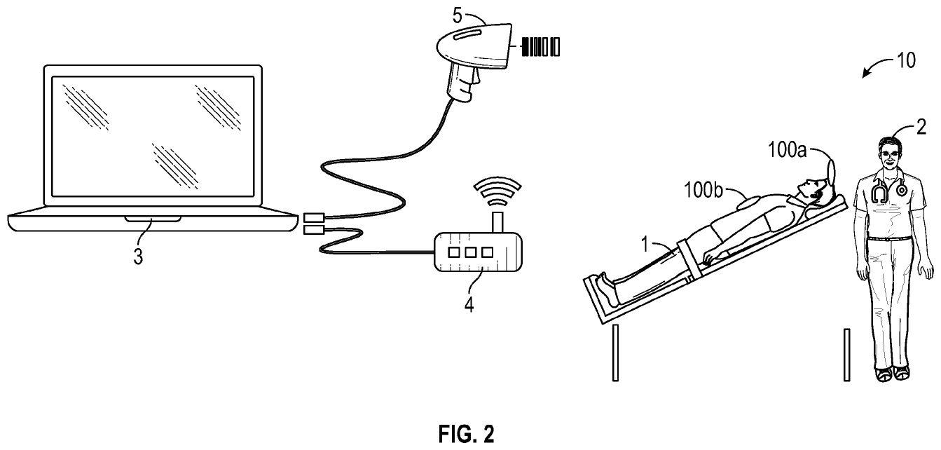

FIG. 2 illustrates an example sensor environment 10. In the environment 10, one or more sensor assemblies 100a, 100b can be implanted by a medical practitioner 2 in the body of a patient 1. The sensor assembly 100a, 100b can include an associated implantable reporting processor (“IPR”) that can be arranged and configured to collect data including for example, medical and health data related to the patient 1 which the sensor assembly 100a, 100b is associated, and operational data of the sensor assembly 100a, 100b itself. The sensor assembly 100a, 100b can communicate with one or more base stations 4 or one or more computing devices 3 during different stages of monitoring the patient 1. While implanted in the patient's body 1, the sensor assembly 100a, 100b can also communicate with a barcode scanner 5 such that the barcode scanner 5 can identify the particular sensor assembly 100a, 100b implanted in the patient 1. The barcode scanner 5 and/or the base station 4 can communicate with the one or more computing devices 3.

For example, in association with a medical procedure, the sensor assembly 100a, 100b can be implanted in the patient's body 1. The sensor assembly 100a, 100b can communicate with an operating room base station 4. While the patient 1 is at home and after sufficient recovery from the medical procedure, the sensor assembly 100a, 100b can be arranged to communicate with a home base station (not shown) and/or a doctor office base station (not shown). The sensor assembly 100a, 100b can communicate with each base station via a short range network protocol, such as the medical implant communication service (“MICS”), the medical device radio communications service (“MedRadio”), industrial, scientific and medical (“ISM”), Bluetooth®, or some other wireless communication protocol suitable for use with the sensor assembly 100a, 100b. MICS band can have frequency range (or bandwidth) of about 402 MHz to 405 MHz, with a center frequency of about 403.5 MHz. ISM band can have bandwidth of about 2.4 GHz to 2.5 GHZ, with a center frequency of about 2.45 GHz. This frequency band can at least partially overlap with the Bluetooth frequency band.

The sensor assembly 100a, 100b may be a standalone medical device or it may be a component in a larger system, including an anchor structure that can desirably collect and provide in situ-patient medical data, device operational data, or other useful data.

The sensor assembly 100a, 100b can include one or more measurement units, for example a sensor, that can collect information and data, including medical and health data related to a patient 1 which the sensor assembly 100a, 100b is associated, and operational data of the assembly 100a, 100b itself.

The sensor assembly 100a, 100b can collect data at various different times and at various different rates during a monitoring process of the patient 1. In some configurations, the sensor assembly 100a, 100b may operate in a plurality of different phases over the course of monitoring the patient. For example, the sensor assembly 100a, 100b can collect more data soon after the sensor assembly 100a, 100b is implanted into the patient 1 and less data as the patient 1 heals and thereafter.

The amount and type of data collected by a sensor assembly 100a, 100b may be different from patient to patient, and the amount and type of data collected may change for a single patient. For example, a medical practitioner studying data collected by the sensor assembly 100a, 100b of a particular patient may adjust or otherwise control how the sensor assembly 100a, 100b collects future data.

The amount and type of data collected by a sensor assembly 100a, 100b may be different for different body parts, for different types of patient conditions, for different patient demographics, or for other differences. Alternatively, or in addition, the amount and type of data collected may change overtime based on other factors, such as how the patient is healing or feeling, how long the monitoring process is projected to last, how much battery power remains and should be conserved, the type of movement being monitored, the body part being monitored, and the like. In some cases, the collected data can be supplemented with personally descriptive information provided by the patient such as subjective pain data, quality of life metric data, co-morbidities, perceptions or expectations that the patient associates with the sensor assembly 100a, 100b, or the like.

Implantation of the sensor assembly 100a, 100b into the patient 1 may occur in an operating room. As used herein, operating room includes any office, room, building, or facility where the sensor assembly 100a, 100b can be implanted into the patient. For example, the operating room may be a typical operating room in a hospital, an operating room in a surgical clinic or a doctor's office, or any other operating theater, interventional suite, intensive care ward, emergency room or the like where the sensor assembly 100a, 100b is implanted into the patient.

The operating room base station 4 can be utilized to configure and initialize the sensor assembly 100a, 100b when the sensor assembly 100a, 100b is being implanted into the patient 1. A communicative relationship can be formed between the sensor assembly 100a, 100b and the operating room base station 4, for example, based on a polling signal transmitted by the operating room base station 4 and a response signal transmitted by the sensor assembly 100a, 100b.

Upon forming a communicative relationship, which can often occur prior to implantation of the sensor assembly 100a, 100b, the operating room base station 4 can transmit initial configuration information to the sensor assembly 100a, 100b. The initial configuration information may include, but is not limited to, a time stamp, a day stamp, an identification of the type and placement of the sensor assembly 100a, 100b, information on other implants associated with the sensor assembly 100a, 100b, surgeon information, patient identification, operating room information, and the like.

In some configurations, the initial configuration information can be passed unidirectionally. In some embodiments, the initial configuration information can be passed bidirectionally. The initial configuration information may define at least one parameter associated with the collection of data by the sensor assembly 100a, 100b. For example, the initial configuration information may identify settings for one or more sensors of the sensor assembly 100a, 100b for each of one or more modes of operation. The initial configuration information may also include other control information, such as an initial mode of operation of the sensor assembly 100a, 100b, a particular event that triggers a change in the mode of operation, radio settings, data collection information (e.g., how often the sensor assembly 100a, 100b wakes up to collected data, how long it collects data, how much data to collect), home base station (not shown), computing device 3, and a connected personal assistant identification information, and other control information associated with the implantation or operation of the sensor assembly 100a, 100b. Examples of a connected personal assistant, which also can be called a smart speaker, include Amazon Echo®, Amazon Dot®, Google Home®, Philips® patient monitor, Comcast's health-tracking speaker, and Apple HomePod®.

In some configurations, the initial configuration information may be pre-stored on the operating room base station 4 or an associated computing device 3. In other configurations, a surgeon, surgical technician, or some other medical practitioner 2 may input the control information and other parameters to the operating room base station 4 for transmission to the sensor assembly 100a, 100b. In at least one such configuration, the operating room base station 4 may communicate with an operating room configuration computing device 3. The operating room configuration computing device 3 can include an application with a graphical user interface that enables the medical practitioner to input configuration information for the sensor assembly 100a, 100b. In various configurations, the application executing on the operating room configuration computing device 3 may have some of the configuration information predefined, which may or may not be adjustable by the medical practitioner 2. The operating room configuration computing device 3 can communicate the configuration information to the operating room base station 4 via a wired, or wireless network connection (e.g., via a USB connection, Bluetooth connection, Bluetooth Low Energy (“BTLE”) connection, or Wi-Fi connection), which can communicate it to the sensor assembly 100a, 100b.

The operating room configuration computing device 3 may also display information regarding the sensor assembly 100a, 100b or the operating room base station 4 to the surgeon, surgical technician, or other medical practitioner 2. For example, the operating room configuration computing device 3 may display error information if the sensor assembly 100a, 100b is unable to store or access the configuration information, if the sensor assembly 100a, 100b is unresponsive, if the sensor assembly 100a, 100b identifies an issue with one of the sensors or radio during an initial self-test, if the operating room base station 4 is unresponsive or malfunctions, or for other reasons.

Although the operating room base station 4 and the operating room configuration computing device 3 are described as separate devices, embodiments are not so limited; rather, the functionality of the operating room configuration computing device 3 and the operating room base station 4 may be included in a single computing device or in separate devices as illustrated. In this way, the medical practitioner 1 may be enabled in one embodiment to input the configuration information directly into the operating room base station 4.

Returning to FIG. 1, once the sensor assembly 100a, 100b is implanted into the patient and the patient returns home, the home base station, the computing or smart device (e.g., the patient's smart phone), the connected personal assistant, or two or more of the home base station, and the computing or smart device, and the connected personal assistant can communicate with the sensor assembly 100a, 100b. The sensor assembly 100a, 100b can collect data at determined rates and times, variable rates and times, or otherwise controllable rates and times. Data collection can start when the sensor assembly 100a, 100b is initialized in the operating room, when directed by a medical practitioner 1, or at some later point in time. At least some data collected by the sensor assembly 100a, 100b may be transmitted to the home base station directly, to the smart device directly, to the connected personal assistant directly, to the base station via one or both of the smart device and the connected personal assistant, to the smart device via one or both of the base station and the connected personal assistant, or to the connected personal assistant via one or both of the smart device and the base station. Here, “one or both” means via an item alone, and via both items serially or in parallel. For example, data collected by the sensor assembly 100a, 100b may be transmitted to the home base station via the smart device alone, via the connected personal assistant alone, serially via the smart device and the connected personal assistant, serially via the connected personal assistant and the smart device, and directly, and possibly contemporaneously, via both the smart device and the connected personal assistant. Similarly, data collected by the sensor assembly 100a, 100b may be transmitted to the smart device via the home base station alone, via the connected personal assistant alone, serially via the home base station and the connected personal assistant, serially via the connected personal assistant and the home base station, and directly, and possibly contemporaneously, via both the home base station and the connected personal assistant. Further in example, data collected by the sensor assembly 100a, 100b may be transmitted to the connected personal assistant via the smart device alone, via the home base station alone, serially via the smart device and the home base station, serially via the home base station and the smart device, and directly, and possibly contemporaneously, via both the smart device and the home base station.

In various configurations, one or more of the home base station, the smart device, and the connected personal assistant can ping the sensor assembly 100a, 100b at periodic, predetermined, or other times to determine if the sensor assembly 100a, 100b is within communication range of one or more of the home base station, the smart device, and the connected personal assistant. Based on a response from the sensor assembly 100a, 100b, one or more of the home base station, the smart device, and the connected personal assistant determines that the sensor assembly 100a, 100b is within communication range, and the sensor assembly 100a, 100b can be requested, commanded, or otherwise directed to transmit the data it has collected to one or more of the home base station, the smart device, and the connected personal assistant.

Each of one or more of the home base station, the smart device, and the connected personal assistant may, in some cases, be arranged with a respective optional user interface. The user interface may be formed as a multimedia interface that unidirectionally or bi-directionally passes one or more types of multimedia information (e.g., video, audio, tactile, etc.). Via the respective user interface of one or more of the home base station, the smart device, and the connected personal assistant, the patient 1 or an associate of the patient 1 may enter other data to supplement the data collected by the sensor assembly 100a, 100b. A user, for example, may enter personally descriptive information (e.g., age change, weight change), changes in medical condition, co-morbidities, pain levels, quality of life, or other subjective metric data, personal messages for a medical practitioner, and the like. In these configurations, the personally descriptive information may be entered with a keyboard, mouse, touch-screen, microphone, wired or wireless computing interface, or some other input means. In cases where the personally descriptive information is collected, the personally descriptive information may include, or otherwise be associated with, one or more identifiers that associate the information with unique identifier of the sensor assembly 100a, 100b, the patient, an associated medical practitioner, an associated medical facility, or the like.

In some of these cases, a respective optional user interface of each of one or more of the home base station, the smart device, and the connected personal device may also be arranged to deliver information associated with the sensor assembly 100a, 100b to the user from, for example, a medical practitioner 2. In these cases, the information delivered to the user may be delivered via a video screen, an audio output device, a tactile transducer, a wired or wireless computing interface, or some other like means.

In configurations where one or more of the home base station, the smart device, and the connected personal assistant are arranged with a user interface, which may be formed with an internal user interface arranged for communicative coupling to a patient portal device. The patent portal device may be smartphone, a tablet, a body-worn device, a weight or other health measurement device (e.g., thermometer, bathroom scale, etc.), or some other computing device capable of wired or wireless communication. In these cases, the user is able to enter the personally descriptive information, and the user also may be able to receive information associated with the sensor assembly 100a, 100b.

The home base station can utilize a home network of the patient to transmit the collected data to cloud. The home network, which may be a local area network, provides access from the home of the patient to a wide area network, such as the internet. In some configurations, the home base station may utilize a Wi-Fi connection to connect to the home network and access the internet. In other embodiments, the home base station may be connected to a home computer (not shown) of the patient, such as via a USB connection, which itself is connected to the home network.

The smart device can communicate with the sensor assembly 100a, 100b directly via, for example, Bluetooth® compatible signals, and can utilize the home network of the patient to transmit the collected data to cloud, or can communicate directly with the cloud, for example, via a cellular network. Alternatively, the smart device can be configured to communicate directly with one or both of the base station and the connected personal assistant via, for example, Bluetooth® compatible signals, and is not configured to communicate directly with the sensor assembly 100a, 100b.

Furthermore, the connected personal assistant can communicate with the sensor assembly 100a, 100b directly via, for example, Bluetooth® compatible signals, and can utilize the home network of the patient to transmit the collected data to cloud, or can communicate directly with the cloud, for example, via a modem/internet connection or a cellular network. Alternatively, the connected personal assistant can be configured to communicate directly with one or both of the base station and the smart device via, for example, Bluetooth® compatible signals, and not configured to communicate directly with the sensor assembly 100a, 100b.

Along with transmitting collected data to the cloud, one or more of the home base station, the smart device, and the connected personal assistant may also obtain data, commands, or other information from the cloud directly or via the home network. One or more of the home base station, the smart device, and the connected personal assistant may provide some or all of the received data, commands, or other information to the sensor assembly 100a, 100b. Examples of such information include, but are not limited to, updated configuration information, diagnostic requests to determine if the sensor assembly 100a, 100b is functioning properly, data collection requests, and other information.

The cloud may include one or more server computers or databases to aggregate data collected from the sensor assembly 100a, 100b, and in some cases personally descriptive information collected from a patient, with data collected from other intelligent implantable devices, and in some cases personally descriptive information collected from other patients. In this way, the cloud can create a variety of different metrics regarding collected data from each of a plurality of intelligent implantable devices that are implanted into separate patients. This information can be helpful in determining if the intelligent implantable devices are functioning properly. The collected information may also be helpful for other purposes, such as determining which specific devices may not be functioning properly, determining if a procedure or condition associated with the intelligent implantable device is helping the patient (e.g., if a sensor system, which includes a sensor assembly 100a, 100b, is operating properly), and determining other medical information.

At various times throughout the monitoring process, the patient may be requested to visit a medical practitioner for follow up appointments. This medical practitioner may be the surgeon who implanted the sensor assembly 100a, 100b in the patient or a different medical practitioner that supervises the monitoring process, physical therapy, and recovery of the patient. For a variety of different reasons, the medical practitioner may want to collect real-time data from the sensor assembly 100a, 100b in a controlled environment. In some cases, the request to visit the medical practitioner may be delivered through a respective optional bidirectional user interface of each of one or more of the home base station, the smart device, and the connected personal assistant.

A medical practitioner can utilize the doctor office base station, which communicates with the sensor assembly 100a, 100b, to pass additional data between the doctor office base station and the sensor assembly 100a, 100b. Alternatively, or in addition, the medical practitioner can utilize the doctor office base station to pass commands to the sensor assembly 100a, 100b. In some configurations, the doctor office base station can instruct the sensor assembly 100a, 100b to enter a high-resolution mode to temporarily increase the rate or type of data that is collected for a short time. The high-resolution mode directs the sensor assembly 100a, 100b to collect different (e.g., large) amounts of data during an activity where the medical practitioner is also monitoring the patient.

In some configurations, the doctor office base station can enable the medical practitioner to input event markers, which can be synchronized with the high-resolution data collected by the sensor assembly 100a, 100b. For example, assume the sensor assembly 100a, 100b is a component in a sensor system adapted to be implanted into a joint. During a follow up visit, the medical practitioner can put the sensor assembly 100a, 100b in the high-resolution mode. The medical practitioner can review the sensor data from the sensor assembly 100a, 100b and determine whether the injury is (or has) healed and the implant is performing correctly. If the sensor data indicates that there is a problem, the medical practitioner can take one or more remedial actions. After the medical practitioner has taken such one or more actions, the medical practitioner can click an event marker button on the doctor office base station to mark performance of such one or more actions. The doctor office base station records the marker and the time at which the marker was input. When the timing of this marker is synchronized with the timing of the collected high-resolution data, the medical practitioner can analyze the data to try and determine the effects of the medication.

In other configurations, the doctor office base station may provide updated configuration information to the sensor assembly 100a, 100b. The sensor assembly 100a, 100b can store this updated configuration information, which can be used to adjust the parameters associated with the collection of the data. For example, if the patient is doing well, the medical practitioner can direct a reduction in the frequency at which the sensor assembly 100a, 100b collects data. On the contrary, if the defect or injury is not healing or the implant is not performing correctly, the medical practitioner may direct the sensor assembly 100a, 100b to collect additional data for a determined period of time (e.g., a few days). The medical practitioner may use the additional data to diagnose and treat a particular problem. In some cases, the additional data may include personally descriptive information provided by the patient after the patient has left presence of the medical practitioner and is no longer in range of the doctor office base station. In these cases, the personally descriptive information may be collected and delivered from via one or more of the home base station, the smart device, and the connected personal assistant. Firmware within the sensor assembly 100a, 100b and/or the base station can provide safeguards limiting the duration of such enhanced monitoring to insure the battery retains sufficient power to last for the implant's lifecycle. Additionally, or alternatively, the sensor assembly 100a, 100b can include a conductive switch, which is further described below, that assists in limiting the monitoring of the sensor assembly 100a, 100b.

In various configurations, the doctor office base station may communicate with a doctor office configuration computing device. The doctor office configuration computing device can include an application with a graphical user interface that enables the medical practitioner to input commands and data. Some or all of the commands, data, and other information may be later transmitted to the sensor assembly 100a, 100b via the doctor office base station. For example, in some configurations, the medical practitioner can use the graphical user interface to instruct the sensor assembly 100a, 100b to enter its high-resolution mode. In other configurations, the medical practitioner can use graphical user interface to input or modify the configuration information for the sensor assembly 100a, 100b. The doctor office configuration computing device can transmit the information (e.g., commands, data, or other information) to the doctor office base station via a wired or wireless network connection (e.g., via a USB connection, Bluetooth® connection, or Wi-Fi connection), which in turn can transmits some or all of the information to the sensor assembly 100a, 100b.

The doctor office configuration computing device may also display, to the medical practitioner, other information regarding the sensor assembly 100a, 100b, regarding the patient (e.g., personally descriptive information), or the doctor office base station. For example, the doctor office configuration computing device may display the high-resolution data that is collected by the sensor assembly 100a, 100b and transmitted to the doctor office base station. The doctor office configuration computing device may also display error information if the sensor assembly 100a, 100b is unable to store or access the configuration information, if the sensor assembly 100a, 100b is unresponsive, if the sensor assembly 100a, 100b identifies an issue with one of the sensors or radio, if the doctor office base station is unresponsive or malfunctions, or for other reasons.

In some configurations, doctor office configuration computing device may have access to the cloud. In at least one embodiment, the medical practitioner can utilize the doctor office configuration computing device to access data stored in the cloud, which was previously collected by the sensor assembly 100a, 100b and transmitted to the cloud via one or both of the home base station and the smart device. Similarly, the doctor office configuration computing device can transmit the high-resolution data obtain from the sensor assembly 100a, 100b via the doctor office base station to the cloud. In some configurations, the doctor office base station may have internet access and may be enabled to transmit the high-resolution data directly to the cloud without the use of the doctor office configuration computing device.

In various configurations, the medical practitioner may update the configuration information of the sensor assembly 100a, 100b when the patient is not in the medical practitioner's office. In these cases, the medical practitioner can utilize the doctor office configuration computing device to transmit updated configuration information to the sensor assembly 100a, 100b via the cloud. One or more of the home base station, the smart device, and the connected personal assistant can obtain updated configuration information from the cloud and pass updated configuration information to the cloud. This can allow the medical practitioner to remotely adjust the operation of the sensor assembly 100a, 100b without needing the patient to come to the medical practitioner's office. This may also permit the medical practitioner to send messages to the patient in response, for example, to personally descriptive information that was provided by the patient and passed through one or more of the home base station, the smart device, and the connected personal assistant to the doctor office base station.

Although the doctor office base station and the doctor office configuration computing device are described as separate devices, configurations are not so limited; rather, the functionality of the doctor office configuration computing device and the doctor office base station may be included in a single computing device or in separate devices (as illustrated). In this way, the medical practitioner may be enabled in one configuration to input the configuration information or markers directly into the doctor office base station and view the high-resolution data (and synchronized marker information) from a display on the doctor office base station.

Still referring to FIG. 2, alternate configurations are contemplated. For example, each of the base station, the smart device, and the connected personal assistant may be configured to communicate with one or both of the sensor assembly 100a, 100b and the cloud via another one or two of the base station, the smart device, and the connected personal assistant. Moreover, the smart device can be temporarily contracted as an interface to the sensor assembly 100a, 100b, and can be any suitable device other than a smart phone, such as a smart watch, a smart patch, and any IoT device, such as a coffee pot, capable of acting as an interface to the sensor assembly 100a, 100b. In addition, one or more of the base station, smart device, and connected personal assistant can act as a communication hub for multiple sensor assemblies 100a, 100b implanted in one or more patients. Furthermore, one or more of the base station, smart device, and connected personal assistant can automatically order or reorder prescriptions or medical supplies (e.g., a calcium channel blocker) in response to patient input or sensor assembly 100a, 100b input (e.g., pain level, level of clotting) if a medical professional and insurance company have preauthorized such an order or reorder; alternatively, one or more of the base station, smart device, and connected personal assistant can be configured to request, from a medical professional or an insurance company, authorization to place the order or reorder. Moreover, one or more of the base station, smart device, and connected personal assistant can be configured with a personal assistant such as Alexa® or Siri®.

### II. Sensor Assembly

Any of the sensor assemblies described herein are capable of being implanted within any joint or, more generally, in a patient's body. Although certain examples described below relate to a hip implant, systems and methods described herein are applicable to an implant for the knee, shoulder, or elbow, implant of another joint, or implantable structure for any body part.

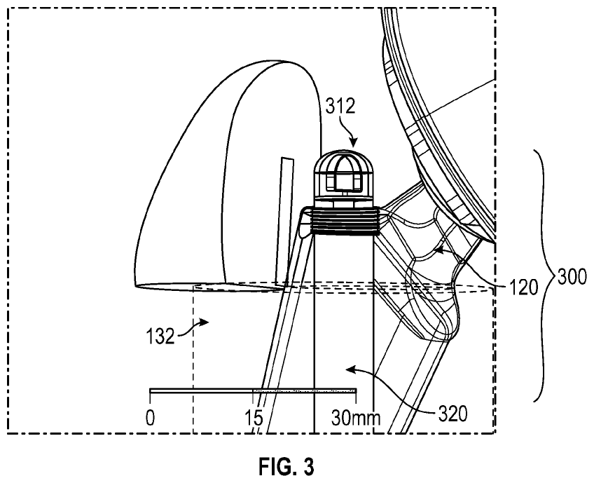

As shown in FIG. 3, a sensor assembly 300 can include an enclosure or housing 320 and an implantable reporting processor with one or more antennas 312 (sometimes referred to in this disclosure in a singular form as an antenna). The housing can enclose electronic circuitry of the implantable reporting processor, which can include at least one of one or more sensors, transmission and receive circuitry, controller, or memory. The transmission and receive circuitry can include one or more of radio-frequency (RF) font-end matching network, one or more filters (such as, one or more surface wave acoustic filters), and a transceiver. As described herein, the one or more sensors can monitor conditions of the patient, such as movement or range of motion. For example, the one or more sensors can include one or more of an accelerometer or gyroscope to monitor whether the patient is standing, sitting, walking, or laying down, monitor the gait, range of motion, or the like. The one or more sensors can include temperature or current sensing. The antenna 312 can transmit data obtained by one or more sensors to a receiver outside the patient's body. For example, the antenna 312 can transmit sensor data continuously, intermittently at a regular time interval, or responsive to receiving a command. The antenna 312 can be connected to the transceiver via a feed. The antenna 312 can be at least partially covered.

Electronic circuitry can be supported by one or more printed circuit boards (PCBs). A power source (such as, one or more batteries) can provide power to the electronic circuitry and the antenna 312. The one or more PCBs and power source can be stacked vertically or horizontally to reduce the size of the sensor assembly 300.

In some instances, the electronic circuitry can operate in a plurality of modes. The electronic circuitry can operate in a first mode, in which little power is consumed, to save power. The first mode can be referred to as a low-power or sleep mode. The electronic circuitry can operate in a second mode, in which at least some components of the electronic circuitry (such as, one or more sensors, controller, etc.) are operational. The second mode can be referred to as an operational mode. In some cases, the electronic circuitry can transition to from the sleep mode into the operational mode responsive to the antenna 312 receiving one or more signals (or commands) in the second frequency band (or, in some cases, in the first frequency band). In the operational mode, the electronic circuitry can, for instance, collect data with the one or more sensors and transmit the data (or any other data) via the antenna 312. The data can be transmitted in the first frequency band (or, in some cases, in the second frequency band). Additionally or alternatively, data can be received via the antenna 312 in the first frequency band (or in the second frequency band). The electronic circuitry can transition from the second mode into the first mode responsive to a receiving data (such as, a command) via the antenna 312, responsive to an expiration of a duration of time, or the like.

The sensor assembly 300 can be implanted in a patient's femur 132. As shown in FIG. 3, the sensor assembly 300 can be positioned within or be supported by the femoral stem 122. As another example, the sensor assembly 300 can be implanted directly in the femur 132, such as in an opening created during hip arthroplasty. The housing 320 can be made from biocompatible material, which can be conductive (such as, one or more of titanium or titanium alloy, stainless steel, cobalt-chromium alloy, or the like).

### A. Antenna

An antenna 312, is used to wirelessly transmit data to a receiver (as well as to receive data from the receiver). Transmitted data can be any data obtained by one or more sensors. It may not be trivial to design the antenna 312. The antenna may be subject to size constraints due to being positioned inside a body. Because the antenna 312 may need to be as small as possible, the antenna 312 or any of the antennas described herein can be an electrically small antenna (which can utilize as ground one or more of a ground plane of the electronic circuitry or the conductive housing 320). The largest dimension of an electrically small antenna may be no more than one-tenth of the wavelength of RF signal received or transmitted by the antenna. In some cases, the height of the antenna 312 may need to be minimal so as to not impinge on any of the ligaments or the like in the hip joint (or another joint or body part). The antenna 312 may need to operate in multiple bands, such as in the MICS band (402-405 MHz) and the ISM band (2.4-2.5 GHZ). MICS band can serve as the first frequency band, and ISM band can serve as the second frequency band. In some implementations, the antenna 312 can receive one or more wake-up commands in the second frequency band to cause the electronic circuitry to transition from the first mode to the second mode. The antenna 312 can transmit data collected by one or more sensors in the first frequency band (as well as receive one or more commands relating to the transmission of data in the first frequency band).

The antenna 312 may need to communicate with a base station over a desired distance, such as at least about 10 feet, about 15 feet, or about 20 feet or more.

To summarize, the antenna 312 for use with a sensor assembly for a hip implant may need to have a very small form factor and operate in at least two frequency bands, such as the MICS and ISM frequency bands. However, some of the design parameters for the antenna 312 may be conflicting. For instance, decreasing the size of the antenna 312 allows the antenna to operate at higher frequencies, but the increased frequency (and shorter wavelength) would cause more loss in the tissue due to increased scattering (particularly in case of the antenna being implanted in a joint and surrounded by a significant layer of bone, muscle, fat, skin, etc.). As another example, decreasing the frequency of operation of the antenna 312 (and increasing the wavelength) would increase the operational distance (or range), but at the expense of making the antenna larger.

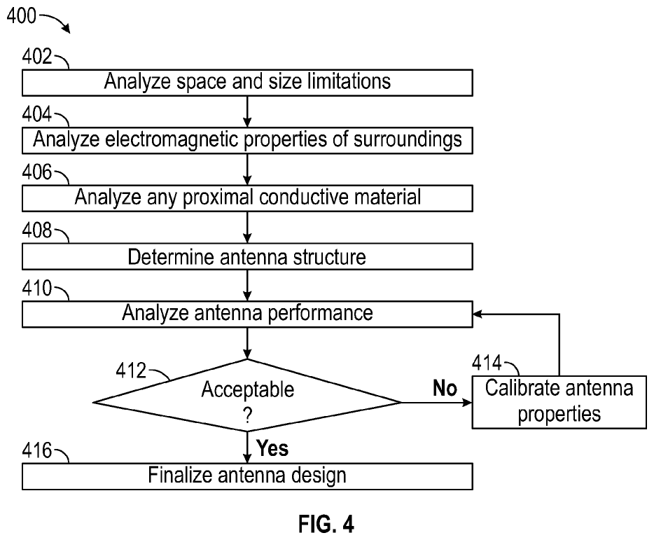

FIG. 4 illustrates a process 400 of an antenna design process, which can be used to design the antenna 312 (or any of the antennas described herein). Prior to designing an antenna, mechanical design constraints may need to be analyzed. Since the size of antenna is related to the antenna performance at particular frequencies, the space where the antenna would be integrated and the antenna size limitation(s) can be analyzed in block 402. After determining the space and antenna size limitations, electromagnetic properties of the material surrounding the antenna (such as, bone, muscle, fat, skin, etc.) can be analyzed in block 404. The electromagnetic properties can include one or more of dielectric constant (or relative permittivity), electrical resistivity, and electrical conductivity, one or more of which can be frequency dependent. The electromagnetic properties of the surrounding material can affect the antenna performance by absorbing energy radiated from (or received by) the antenna. Conductivity of the surrounding material that can affect the antenna performance can be considered in block 406 since material with high electrical conductivity can absorb energy radiated from (or received by) the antenna. For instance, since metal has high electrical conductivity, presence and conductivity of any metal proximal to the antenna can be analyzed in block 406.

Based on the analysis in blocks 402-406, a suitable antenna structure may be determined in block 408. As described herein, the antenna structure can a loop antenna or helical (helix) antenna. The antenna structure can be a substrate antenna (sometimes referred to as a conformal antenna), which can be positioned (such as, printed, painted, or otherwise deposited) on one or more substrate layers. In some cases, the substrate antenna can be a planar inverted-F antenna (PIFA), a meander (or meandered) monopole antenna, or a slot antenna.

After designing the antenna structure in block 408, performance of the antenna can be analyzed in block 410. Antenna performance can be characterized in terms of one or more of gain, bandwidth, radiation pattern, beam width, polarization, impedance, range, or the like. As illustrated in block 412, antenna performance should be within an acceptable range for the particular application. If the performance is not satisfactory, antenna properties can be calibrated as shown in block 414. For example, the structure of antenna can be adjusted by changing the shape or pattern of the antenna. As another example, the antenna can be tuned to resonate at one or more desired frequencies or match its impedance to a desired impedance (such as, the impedance of a transceiver). Blocks 410 and 414 and 406 can be repeated until the antenna exhibits satisfactory performance. The process 400 can terminate in block 416 where antenna design can be finalized. Subsequently, impedance matching can be performed.

A software tool can be used to design and verify the antenna (such as, to model radiation patterns, determine gains, determine return loss, impedance or the like). The software tool can be a finite element method solver for three-dimensional electromagnetic structures, such as such as High Frequency Structure Simulator (HFSS) from Ansys, Inc.

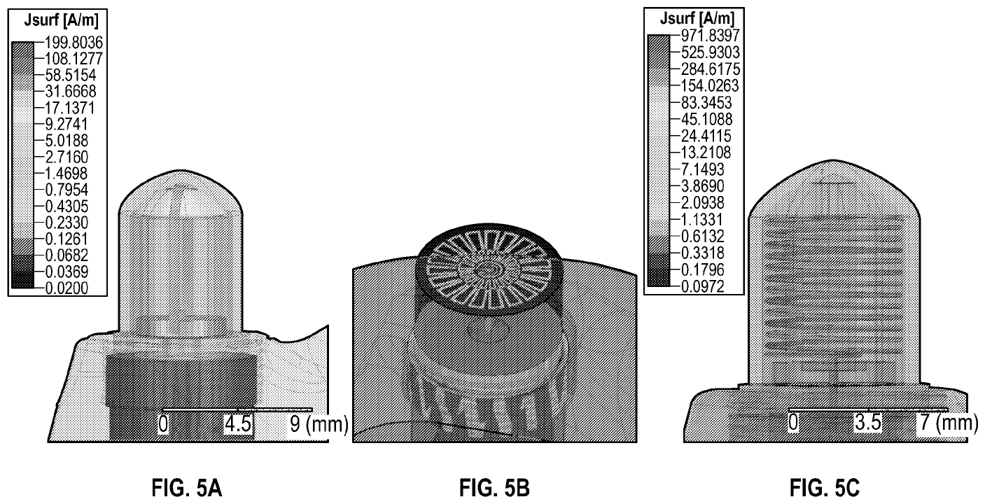

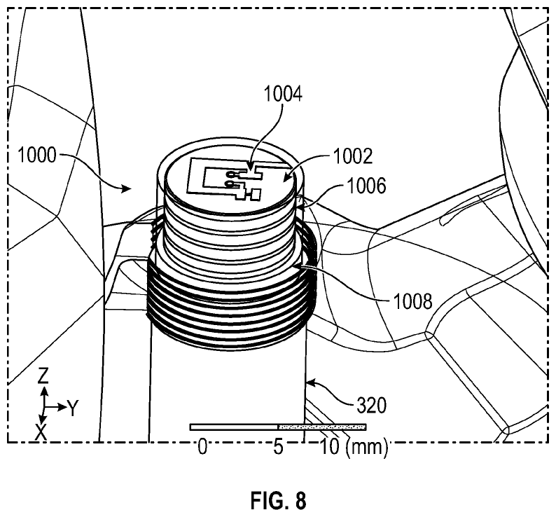

As shown for instance in FIGS. 5A-5C, the antenna 312 can be designed into various types and shapes. For instance, a loop antenna (FIG. 5A), a substrate antenna (FIG. 5B), PIFA antenna (FIG. 8), or a helix antenna (FIG. 5C) may be used. Design and performance of these and other antennas is described below.

a. Loop Antenna

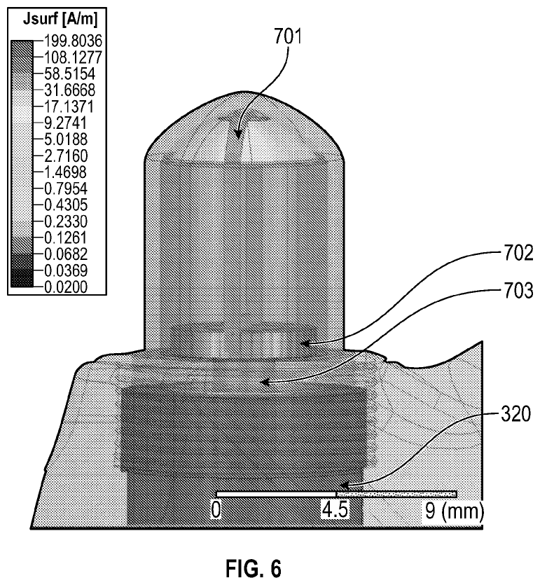

In some embodiments, a loop antenna 701 can be used with the sensor assembly 300. FIG. 6 illustrates a cross section view of the sensor assembly 300 comprising a loop antenna 701 supported by a base 702 (which can be made of conductive material and serve as a ground for the loop antenna 701). A feed 703 to the loop antenna 701 is also illustrated. The feed 703 can connect the loop antenna to electronic circuitry (not shown in FIG. 6), which can include a transmission and receive circuitry. An antenna impedance matching circuitry can connect the feed 703 and a transceiver. The loop antenna 701 can be designed to operate the antenna in the MICS and ISM bands.

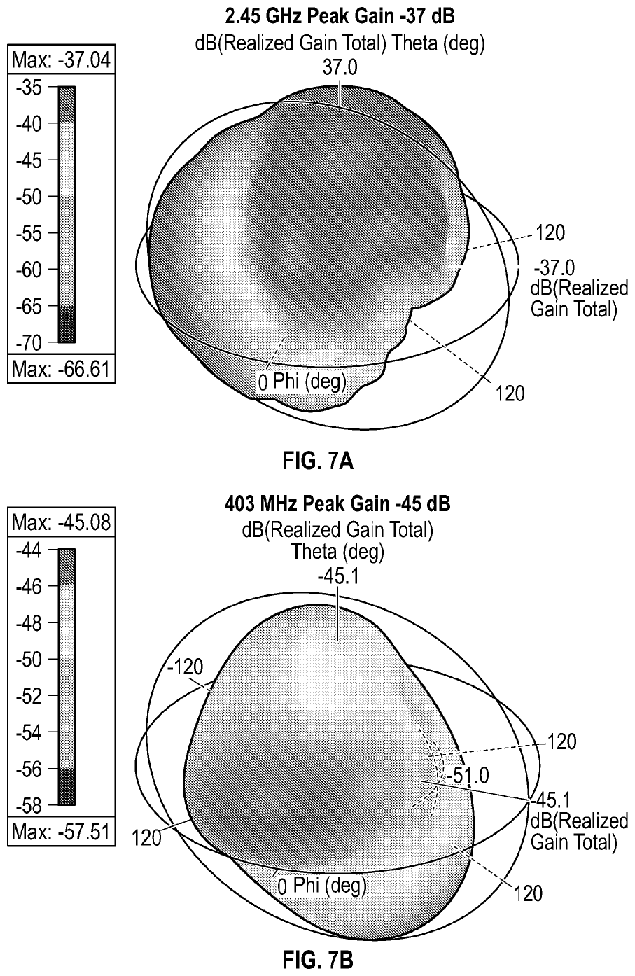

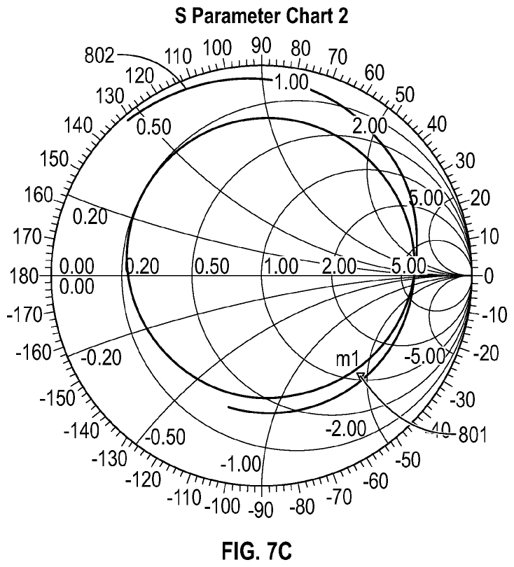

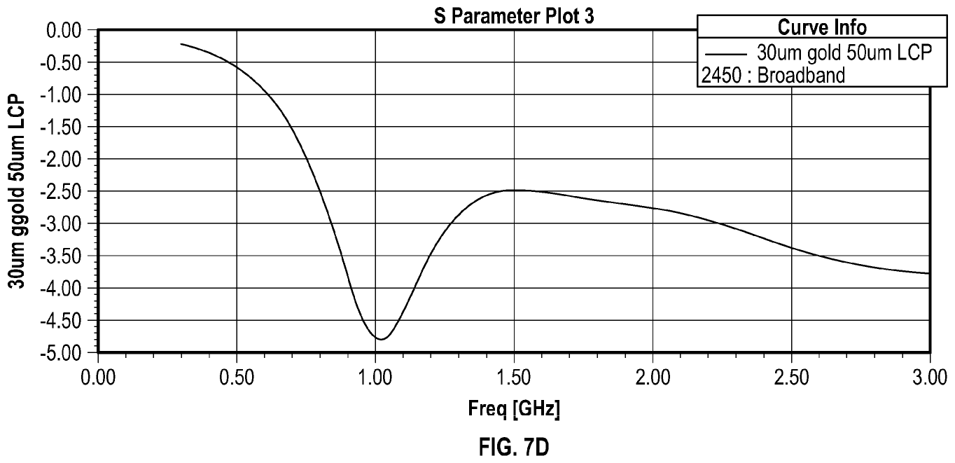

FIGS. 7A and 7B illustrates gain radiation patterns of the loop antenna 701 in the ISM and MICS bands, respectively. As shown in FIGS. 7A and 7B, the loop antenna 701 has a peak gain of −37 decibels (dB) in the ISM band and −45 dB in the MICS band. FIG. 7C shows impedance of the loop antenna 701 plotted on a Smith chart. The impedances 801 and 802 at the center frequencies of the MICS and ISM bands, respectively, are within the Smith chart. Matching network circuitry can be used to adjust the impedances 801 and 802 to match the impedance of the transceiver (such as, 50 Ohm point at the center of the Smith chart).

FIG. 7D illustrates return loss (or S11 parameter) of the loop antenna 701 plotted as a function of frequency. Return loss may be a measure of how much power is reflected from the antenna due to the mismatch from a transmission line. Return loss can be indicative of power not delivered to the antenna (and indicate the amount of power radiated by the antenna). A smaller return loss (such as, about −2 dB, about −3 dB, about −4 dB, about −5 dB, about −10 dB or smaller) can be preferable as it would indicate that a significant amount of energy has been delivered to the antenna. In some cases, target return loss can be about −5 dB or less or around-10 dB or less. FIG. 7D illustrates that the loop antenna 701 resonates at a single frequency of about 1 GHZ, and not at the desired center frequencies of 403.5 MHz in the MICS band and 2.45 GHz in the ISM band.

b. PIFA Antenna

A PIFA antenna can be used with the sensor assembly 300. FIG. 8 depicts a PIFA antenna 1000 supported by a substrate 1002. The PIFA antenna 1000 can include conductive traces 1004 positioned (such as, printed, painted, or otherwise deposited) on the substrate 1002. Conductive traces 1004 can be made of material (such as, one or more of gold, silver, platinum, graphite, copper, etc.) that can be positioned on the substrate 1002. The material can be biocompatible. In some cases, the material can be conductive ink. The PIFA antenna 1000 can be a type of substrate antenna.

The substrate 1002 can be made out of non-conductive biocompatible material, such as, liquid crystal polymer (LCP), polyimide, or polyamide, on which conductive traces can be positioned. Biocompatibility can indicate that a material (such as, the substrate material, conductive traces material, or other biocompatible material described herein), when coming into contact with a tissue, fluids, or generally chemicals in the body, would not cause harm to the body or interact negatively with the living tissue. Biocompatibility can indicate that the material will not trigger an immune response, such as inflammation, irritation, or toxicity. Biocompatible materials can be chemically inert. Biocompatibility can encompass biostability, which indicates the effect on the material from being exposed to the tissue, fluids, or generally chemicals in the body. A biostable material will generally not react with the chemicals in the body. The material of the substrate 1002 can have long-term biocompatibility or biostability (such as, one year or longer) so that the material would not degrade or break down when being exposed to liquid or chemicals in the body. Due to its long-term biostability, LCP can be a preferable material for the substrate 1002. In some implementations, the substrate 1002 can be made out of non-biocompatible material and be coated with biocompatible coating. Non-biocompatible substrate can be encapsulated in biocompatible coating, such as polytetrafluoroethylene (PTFE) coating, fluorinated ethylene propylene (FEP) coating, parylene, acrylated urethane, or combination thereof.

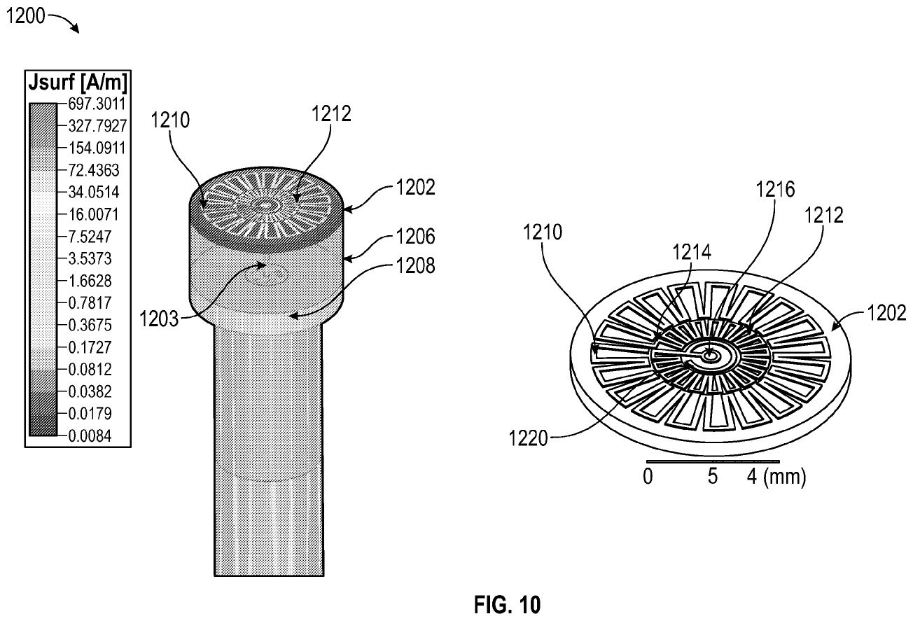

The substrate 1002 can be supported by a spacer 1006 (or a support), which can separate the traces 1004 from the housing 320 of the sensor assembly. The spacer 1006 can be made of non-conductive material (such as, thermoplastic) and can serve as a separator between conductive material of the housing 320 and the conductive antenna traces to improve the antenna performance. The spacer 1006 can be made out of a biocompatible material (such as, polyetheretherketone or PEEK or another biocompatible plastic). As described herein (for instance, in connection with block 406), varying the height of the spacer 1006 can affect the properties of the antenna 1000. To increase the electrical length of the PIFA antenna 1000 (so that antenna can resonate in one or more desired frequency bands), antenna traces can be wound around a spacer 1006 as illustrated in FIG. 8. The spacer 1006 can be supported by a base 1008 (which can be similar to the base 702). A feed (not shown in FIG. 10) can connect the PIFA antenna 1000 to electronic circuitry (not shown in FIG. 10), which can include a transmission and receive circuitry. An antenna impedance matching circuitry can connect the antenna feed and a transceiver. Although the PIFA antenna 1000 is illustrated as a circular structure, the antenna may be non-circular in some implementations.

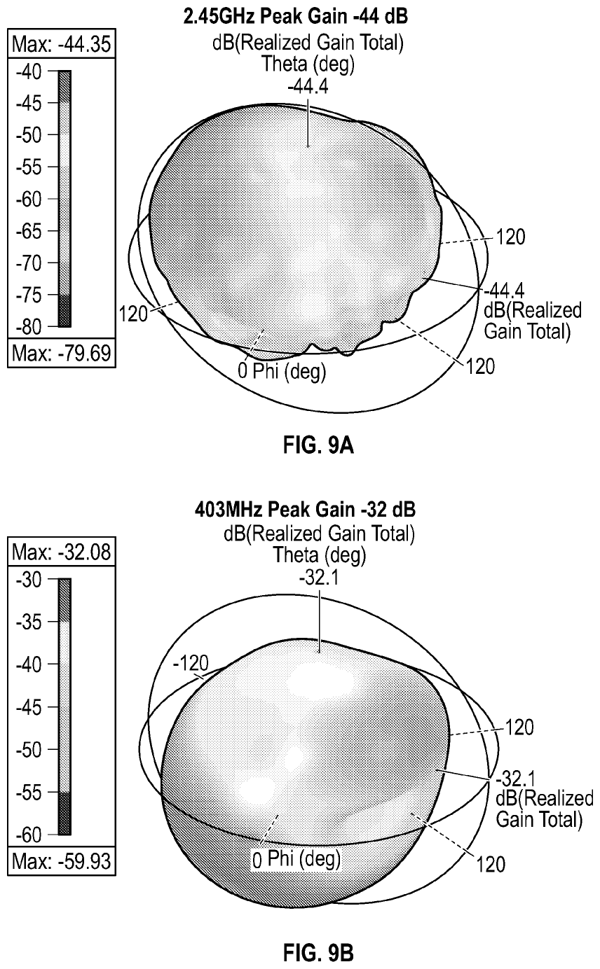

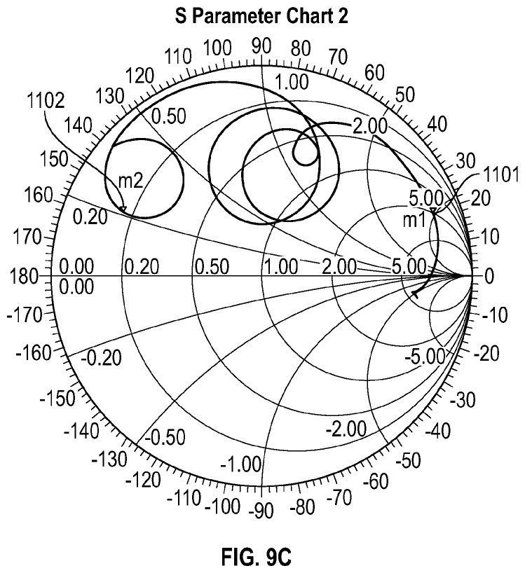

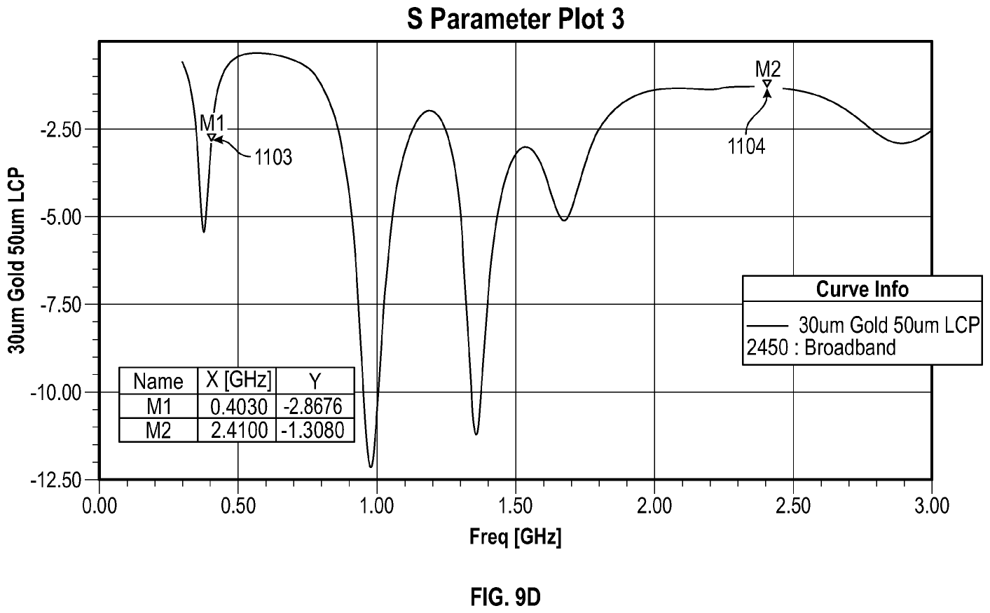

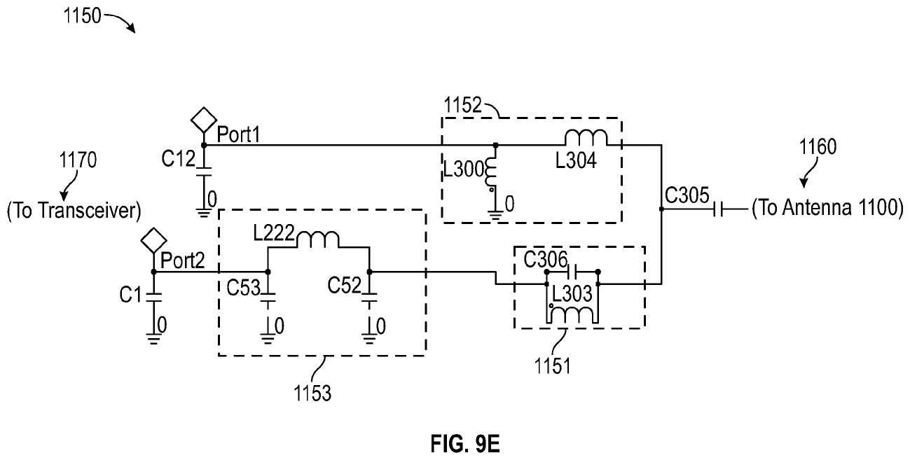

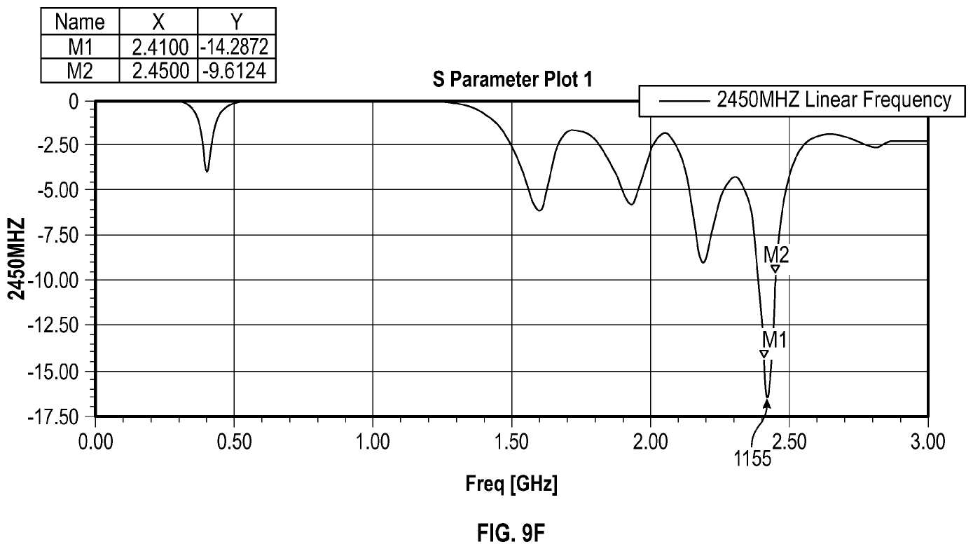

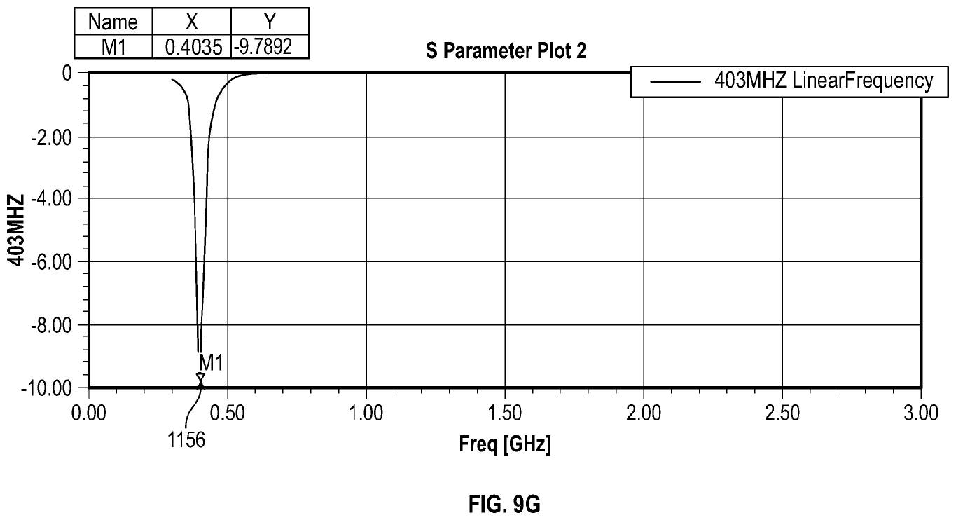

FIGS. 9A and 9B illustrates gain radiation patterns of the PIFA antenna 1000 in the ISM and MICS bands, respectively. As shown in FIGS. 9A and 9B, the PIFA antenna 1000 has a peak gain of −44 dB in the ISM band and −32 dB in the MICS band. FIG. 9C shows impedance of the PIFA antenna 1000 plotted on a Smith chart. Impedances 1101 and 1102 at the center frequencies of the MICS and ISM bands, respectively, are within the Smith chart. Matching network circuitry can be used to adjust the impedances 1101 and 1102 to match the impedance of the transceiver (such as, 50 Ohm point at the center of the Smith chart).

FIG. 9D illustrates return loss of the PIFA antenna 1000 plotted as a function of frequency. As is shown, the PIFA antenna 1000 resonates at many frequencies (such as, about 400 MHz, about 1 GHz, about 1.4 GHz, and about 1.7 GHZ). This performance may not be desirable. For instance, presence of multiple resonant frequencies and narrow bandwidth can make it challenging to tune the PIFA antenna 1000 to operate in the MICS and ISM frequency band and provide an adequate operational range. Return loss at the 403.5 MHz center frequency of the MICS band (illustrated by 1103) is about −2.8 dB. Return loss at the 2.45 GHz center frequency of the ISM band (illustrated by 1104) is about −1.3 dB. These return losses (particularly in the ISM band) may be inadequate (such as, indicative of an insufficient amount of radiated power) for the PIFA antenna 1000 to operate in the MICS and ISM frequency bands and provide an adequate operational range.

FIG. 9E illustrates matching network circuitry 1150 (or matching circuitry) for the PIFA antenna 1000, optimized for peak gain in the ISM and MICS frequency band. The PIFA antenna 1000 can be tuned to improve performance in one or more frequency bands of interest. The matching circuitry 1150 can be designed and connected to the PIFA antenna 1000 to provide one or more of reception or transmission in the first frequency band (such as the MICS band) and in the second frequency band (such as the ISM band). As described herein, the matching circuitry can be designed to account for the dielectric parameters of the tissue surrounding the sensing attachment.

The matching circuitry 1150 can be electrically connected to the PIFA antenna 1000. The matching circuitry can provide impedance matching between the antenna and a transceiver. FIG. 9E illustrates matching circuitry 1150 for the PIFA antenna 1000. The matching circuitry 1150 can include a bottom part designed to process signals in the first frequency band and a top part designed to process signals in the second frequency band. Port 1170 can be connected to the transceiver, which can operate in the first and second frequency bands. In some cases, a filter can be interposed between the PIFA antenna 1000 and the transceiver. For example, the filter can be connected to the port 1170.

A matching network 1152 can process signals in the second frequency band. The matching network 1152 can be inductive. The matching network 1152 is illustrated as an L-network that includes two inductors (such as, a shunt inductor L300 and a series inductor L304). The matching network 1152 can be designed as illustrated in order to match (or counter) the capacitive reactance of the PIFA antenna 1000 in the second frequency band (such as, at higher frequencies).

A band-stop (or notch) filter 1151 can be used to remove higher frequency components from the signals in the first frequency band. The notch filter can remove one or more signal components in the second frequency band from the signals being received (or transmitted) in the first frequency band. The notch filter 1151 is illustrated as a combination of an inductor L303 connected in parallel with capacitor C306. A notch filter may not be included in the matching network for the second frequency band in order to avoid or reduce undesirable parasitic effects (such as, one or more parasitic capacitance or inductance) at the higher frequencies of the second frequency band.

A matching network 1153 can process signals in the first frequency band. The matching network 1153 can be capacitive. The matching network 1153 is illustrated as a Pi-network that includes two capacitors (such as, shunt capacitors C52 and C53 and a series inductor L222). The matching network 1153 can be designed as illustrated in order to match (or counter) the inductive reactance of the PIFA antenna 1000 in the first frequency band (such as, at lower frequencies). Outputs of the top and bottom parts of the matching circuitry 1150 can be connected to the PIFA antenna 1000. In some cases, an output port 1160 can connect the output of the PIFA antenna 1000 to a network analyzer (such as a vector network analyzer (VNA)). The network analyzer can be used for measuring the s-parameters (such as, S11 parameters) of the PIFA antenna 1000. FIG. 9F illustrates the return loss (or S11 parameter) of the PIFA antenna 1000 connected to an optimized ISM band matching circuitry (such as, the matching circuitry 1150). As is shown, the PIFA antenna 1000 resonates at several frequencies (such as, about 400 MHZ, about 1.6 GHz, about 1.94 GHz, and about 2.91 GHz). Return loss at the 2.45 GHz center frequency of the ISM band (illustrated by 1155) is about −16.5 dB. Comparing to FIG. 9D, the addition of an optimized ISM band matching circuitry (such as, the matching circuitry 1150) to the PIFA antenna 1000 can vastly improve the antenna performance (particularly in the ISM band).

FIG. 9G illustrates the return loss (or S11 parameter) of the PIFA antenna 1000 connected to an optimized MICS band matching circuitry (such as, the matching circuitry 1150). As is shown, the PIFA antenna 1000 return loss at 403.5 MHz center frequency of the MICS band (illustrated by 1156) is about −9.8 dB. Comparing to FIG. 9D, the addition of an optimized MICS band matching circuitry (such as, the matching circuitry 1150) to the PIFA antenna 1000 can improve the antenna performance (particularly in the MICS band).

c. Substrate Antenna

As illustrated in FIG. 10, a substrate antenna 1200 can be used with the sensor assembly 300. Similar to the PIFA antenna 1000, the substrate antenna 1200 can include conductive traces positioned (such as, printed, painted, or otherwise deposited) on a substrate 1202 (which can be similar to the substrate 1002). The substrate 1202 can be made of non-conductive, long-term biocompatible material, such as, liquid crystal polymer (LCP), polyimide, or polyamide. In some cases, another layer (or seed layer) can be positioned on the substrate 1202 to facilitate adhesion of the conductive traces (which can be also be applicable for the PIFA antenna 1000). Such layer can be made of titanium. The substrate 1202 can be supported by a spacer 1206 (which can be similar to the spacer 1006) supported by a base 1208 (which can be similar to the base 702 or 1008). The spacer 1206 can be made out of a biocompatible material (such as, PEEK or another biocompatible plastic). A feed 1203 can connect the substrate antenna 1200 to electronic circuitry (not shown in FIG. 10), which can include a transmission and receive circuitry. An antenna impedance matching circuitry can connect the antenna feed 1203 and a transceiver. The substrate antenna 1200 can be referred to as a single-layer substrate antenna since the conductive traces are positioned only on one layer of the substrate 1202. In some implementations, as described herein, a substrate antenna can include conductive traces positioned on multiple layers of the substrate 1202 (such as, the top layer and the bottom layer).

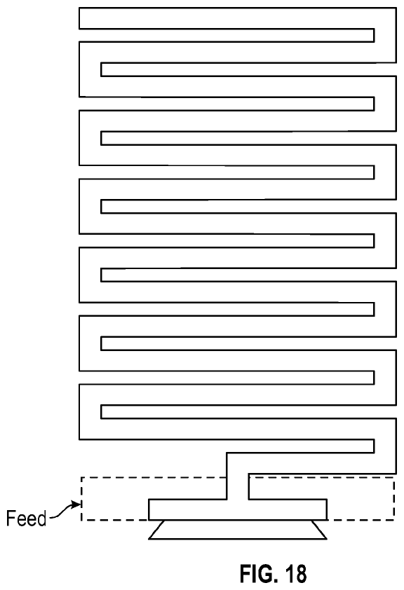

The conductive traces can be arranged into a set of outer traces 1210 and a set of inner traces 1212. The two sets of traces 1210 and 1212 can be connected at 1214. The set of inner traces 1212 can be connected to the feed 1203 at a feed point 1216 positioned at the center of the circle. The set of outer traces (or outer ring) 1210 can include plurality of sections shaped as petals, which can be connected to one another. Similarly, the set of inner traces (or inner ring) 1212 can include a plurality of sections shaped as petals, which can be connected to one another. Shaping the traces as petals can result in a design in which traces are symmetrically arranged around the feed point 1216, which has been found to improve the properties of the substrate antenna 1200. Spacing of the petals in one or more sets of traces 1210 or 1212 can affect the resonance of the substrate antenna 1200. For instance, positioning the petals closer together can improve resonance of the substrate antenna 1200 in one or more of the MICS or ISM bands. Adding more petals to the one or more sets of traces 1210 or 1212 can increase the electrical length of the substrate antenna 1200 and improve resonance in one or more of the MICS or ISM bands. Rounding the corners, such as corners that are shaped like a “V,” can affect the resonance of the substrate antenna 1200. Although petal-shaped sections are illustrated in FIG. 10, other symmetrical (or non-symmetrical) shapes can be used (for example, as described in connection with FIG. 18). For instance, a zig-zag pattern of traces can be used, as shown in FIG. 18).

The substrate antenna 1200 can include a stub 1220, which can be a round trace connected to the set of inner traces 1212. As described herein, varying the length of the stub 1220 can improve the performance of the substrate antenna 1200.

Although the substrate antenna 1200 is illustrated as circular structure, the antenna may be non-circular in some implementations.

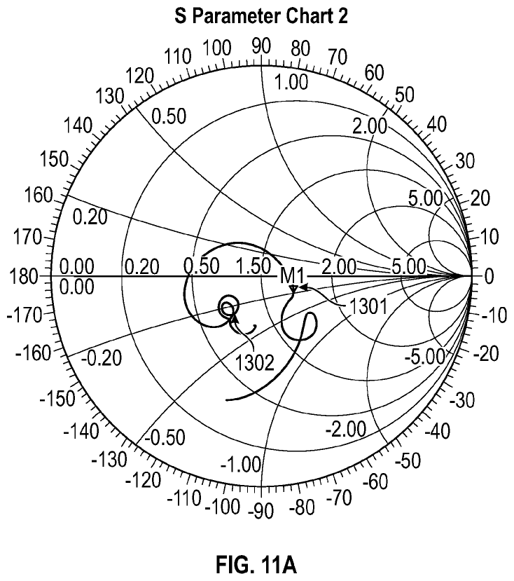

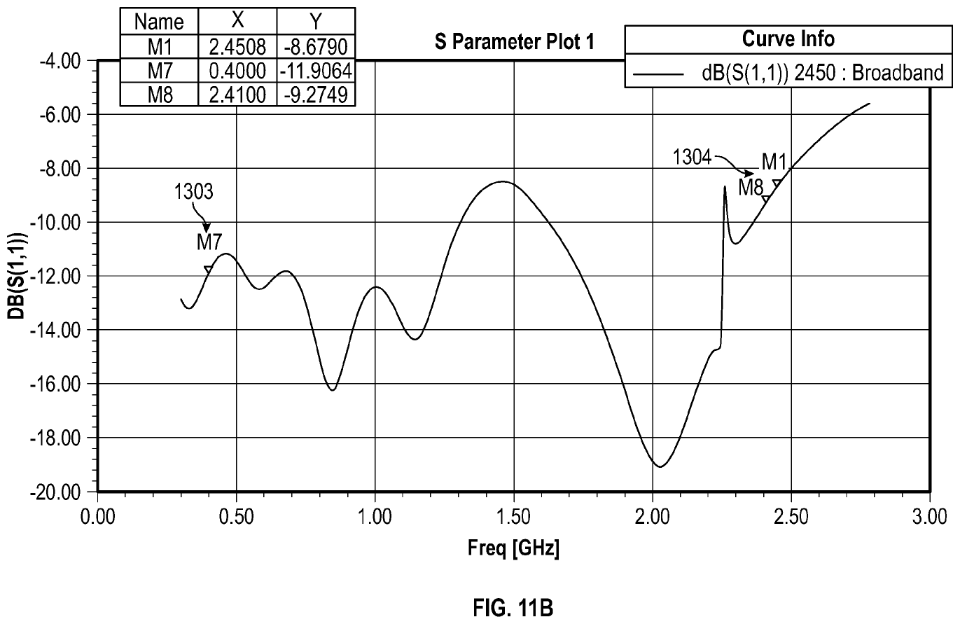

FIG. 11A illustrates impedance of the substrate antenna 1200 plotted on a Smith chart. Impedances 1301 and 1302 at the center frequencies of the MICS and ISM bands, respectively, are within the Smith chart. Matching network circuitry can be used to adjust the impedances 1301 and 1302 to match the impedance of the transceiver (such as, 50 Ohm point at the center of the Smith chart). FIG. 11B illustrates return loss of the substrate antenna 1200 plotted as a function of frequency. As is shown, return loss at the 403.5 MHz center frequency of the MICS band (illustrated by 1303) is about −11.8 dB. Return loss at the 2.45 GHz center frequency of the ISM band (illustrated by 1304) is about −8.7 dB. Advantageously, these return losses indicate that loss of power due to reflections is low for the substrate antenna 1200.

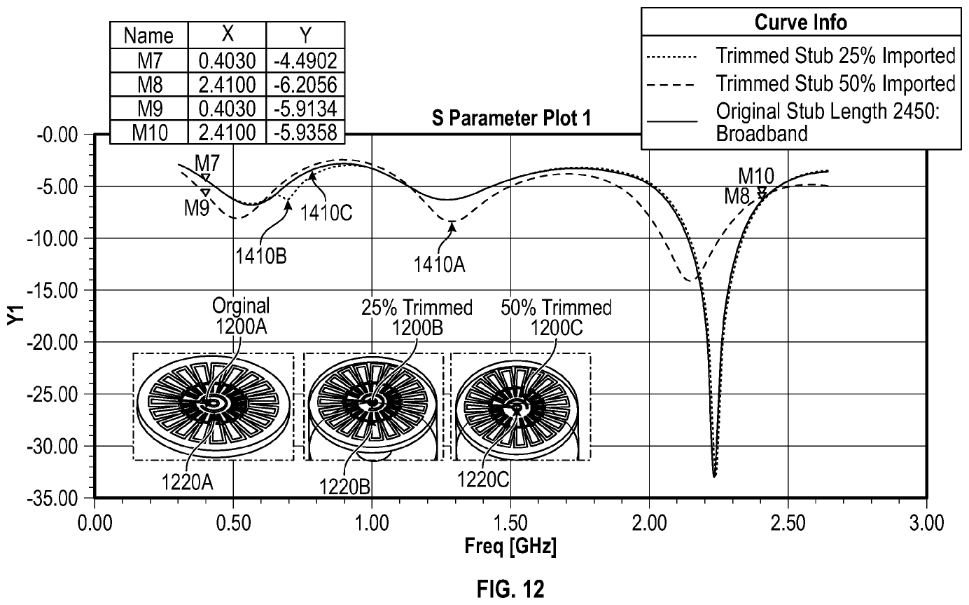

The antenna 1200 can be tuned to improve performance. Tuning can involve varying the length of the stub 1220. FIG. 12 illustrates return loss plotted as a function of frequency for different lengths of the stub 1220. Specifically, the substrate antenna 1200A has a full-length stub 1220A (corresponding to the stub 1220 of the substrate antenna 1200 in FIG. 10). Substrate antenna 1200B has a stub 1220B with the length trimmed by 25% as compared to the stub 1220A. Substrate antenna 1200C has a stub 1220C with the length trimmed by 50% as compared to the stub 1220A. Plot 1410A shows return loss of the antenna 1200A. Plot 1410B shows return loss of the antenna 1200B. Plot 1410C shows return loss of the antenna 1200C. As is illustrated, trimming the stub by 25% and 50% shifts the resonance frequency in the higher frequency band closer to the 2.45 GHz frequency of the ISM band as well as lowers the return loss in the ISM band.

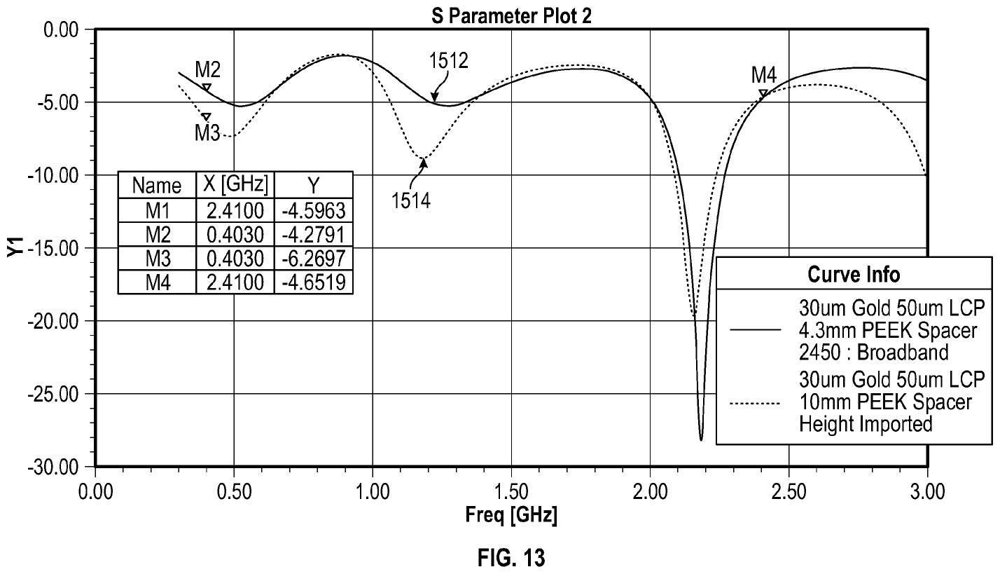

Tuning can involve varying the height of the spacer 1206. Increasing the height of the spacer can improve performance of the antenna 1200, which may be due to moving antenna away from the conductive housing 320. FIG. 13 illustrates return loss plotted as a function of frequency for different heights of the spacer 1206. Plot 1512 illustrates return loss of the antenna 1200 with a shorter spacer 1206 (for example, about 4.3 mm). Plot 1514 illustrates return loss of the antenna 1200 with a longer spacer 1206 (for example, about 10 mm). As is illustrated, resonance of the antenna and gain improve in the MICS band with longer spacer 1206.

Traces of a substrate antenna can be positioned on the top and bottom layers of the substrate 1202 (for example, by deposition, such as by being printed or painted). For instance, the sets of traces 1210 and 1212 can be positioned separately on opposite layers of the substrate 1202. In such arrangement, the sets of traces 1210 and 1212 can be connected by one or more conductive vias, as described herein. For instance, a conductive via can connect two sets of traces 1210 and 1212.

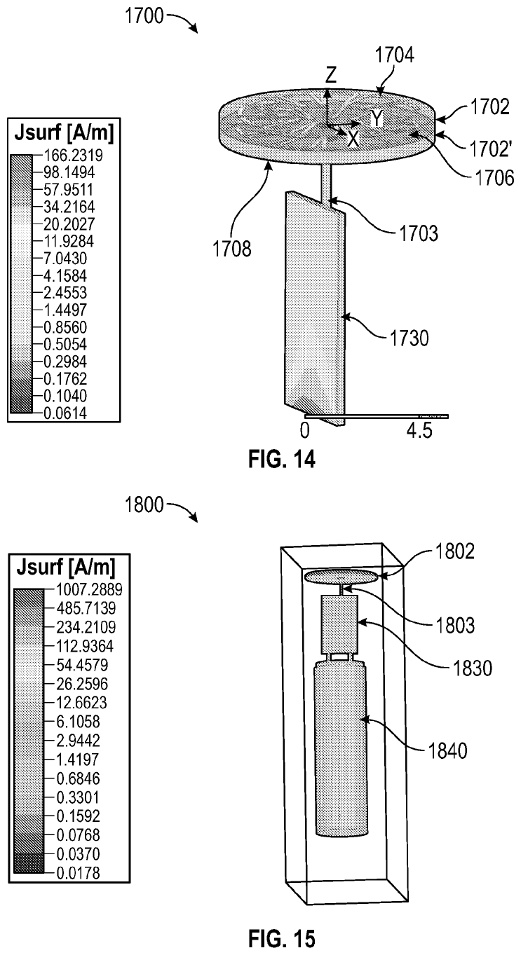

Traces of a substrate antenna can be positioned (such as, printed, painted, or otherwise deposited) on multiple layers of one or more substrates or on multiple substrates. FIG. 14 illustrates a substrate antenna 1700 that includes two substrates 1702 and 1702′ supporting conductive traces 1704 and 1704′. For example, conductive traces 1704 can provide operation in the MICS band and conductive traces 1704′ can provide operation in the ISM band (or vice versa). The conductive traces 1704 and 1704′ can be connected by one or more conductive vias 1706, which can include conductive biocompatible material (which can be the same material as that of the conductive traces). In some cases, a single via 1706 can connect conductive traces 1704 and 1704′. As is illustrated, the via 1706 goes through the substrate 1702. In some implementations, more than one via can be used when more than two substrates are used. The substrates 1702 and 1702′ can be similar to the substrate 1202. The substrate antenna 1700 can be a three-layer antenna with conductive traces being positioned on top and bottom layers of the substrate 1702 and on top (or bottom) layer of the substrate 1702′. A feed 1703 (which can be similar to the feed 1203) can connect the conductive traces to electronic circuitry 1730, which can include a transmission and receive circuitry. In some cases, a substrate antenna can have four or more layers.

Layer 1708 can include conductive material that forms a ground plane. Such ground plane may improve the gain of the antenna by facilitating reflections of the RF energy. Ground plane can similarly positioned (such as, printed, painted, or otherwise deposited) on the opposite side of the substrate 1202 in FIG. 10. Conductive traces can be connected to the ground plane via a via or the feed 1703.

As stated above, any of the antennas described herein can be grounded. Proper grounding can be important for electrically small antennas. FIG. 15 illustrates grounding of a substrate antenna 1800 positioned on a substrate 1802 (which can be similar to the substrate 1202). Although the illustrated antenna is a substrate antenna, any antenna described herein can be similarly grounded. The substrate antenna 1800 can be connected to ground through a feed 1803 (which can be similar to the feed 1203) to a ground plane of the electronic circuitry 1830. The ground plane of the electronic circuitry 1830 can be tied to one or more of a power source 1840 and housing (not illustrated). As a result, the substrate antenna 1800 can be connected to a large ground plane (which can be floating). This can be used to shape the radiation pattern and improve the gain of the antenna 1800.

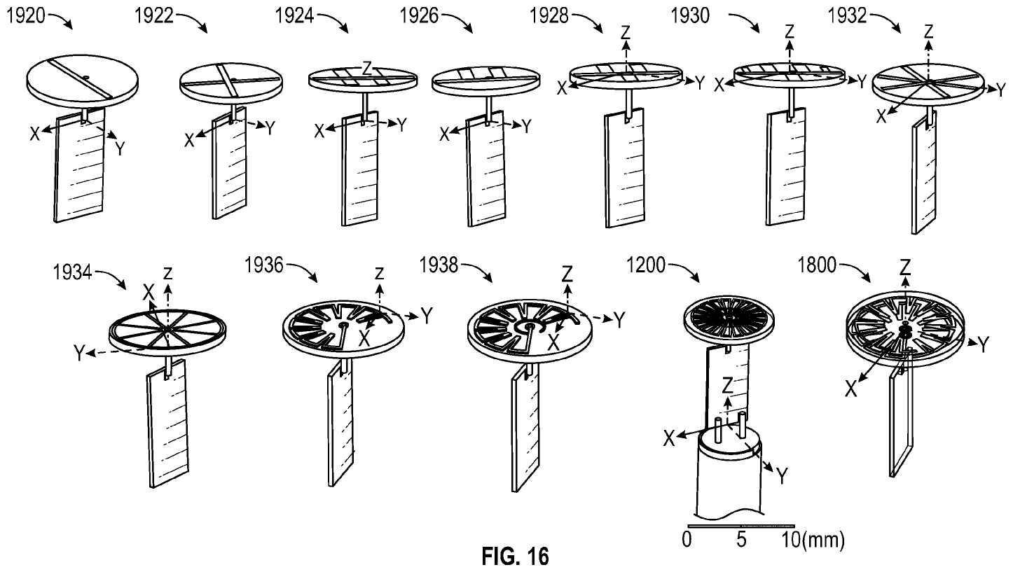

A substrate antenna can have various shapes as illustrated in FIG. 16. Conductive traces can be arranged on a substrate (such as, the substrate 1202) in a pattern 1920 (two wedges) to form a single slot antenna or in a pattern 1922 (four wedges) to form a cross (or T) antenna. Adjusting one or more grounding pins can change polarization of the substrate antenna. Conductive traces can be arranged on the substrate in patterns 1924, 1926, 1928, or 1930. Patterns 1924, 1926, 1928, and 1930 can correspond to variations of U shapes and Pi shapes with the goal of obtaining dual resonance. Conductive traces can be arranged on the substrate, for instance, as six wedges (1932), eight wedges (1934), or the like. The wedges can be of the same size (such as, 1920 and 1922) of different sizes (such as, 1932). Conductive traces can be arranged on the substrate as an incomplete set of petals (1936, 1938) with a stub. As described herein, additional petals can be added to the inner and outer rings to increase the electrical length of the substrate antenna 1200 and obtain the desired resonance properties. A multi-layer petal-shaped substrate antenna 1800 can be designed. In some instances, a single-layer substrate antenna 1200 can be designed by folding petals from one of the layers of the substrate in a dual-layer configuration onto a single layer of the substrate to obtain the inner and outer rings. Advantageously, a single-layer substrate antenna may be easier and cheaper to manufacture than a multi-layer substrate antenna.

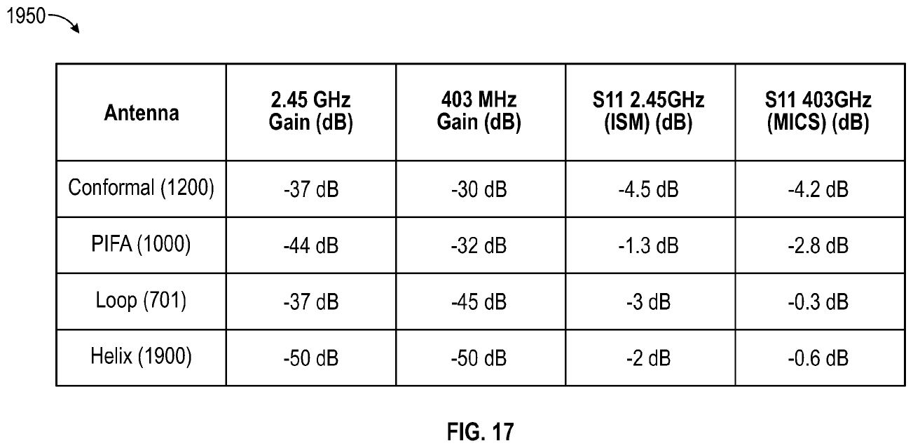

FIG. 17 illustrates a table 1950 comparing performance of various antennas described herein, including the substrate antenna 1200, the helix antenna 1900, the loop antenna 701, and the PIFA antenna 1000. Performance of various antennas has been simulated (for example, using a 3D finite element method solver) taking into account surrounding conductive components, such as one or more of the electronic circuitry, power source, housing, or implant. Performance of various antennas described herein has been simulated not just in free space, but also taking into account the muscle, bone, fat, skin, or the like of the body part where the antenna would be deployed. Frequency dependent relative permittivity (Er) and conductivity (Sigma) of such one or more tissues can be taken into accounting during simulation.

For example, the following relative permittivity and conductivity can be used to test the performance of various antennas:

| | 403 MHz | | 2.45 GHz | |
|---|---|---|---|---|
| | Er | Sigma | Er | Sigma |
| Skin | 40 | 0.4 | 31 | 0.8 |
| Fat | 5.5806 | 0.041088 | 5.2737 | 0.10725 |
| Muscle | 57.144 | 0.79582 | 52.654 | 1.7812 |
| Bone Cortical | 13.152 | 0.091168 | 11.345 | 0.40645 |
| Bone Marrow | 5.6741 | 0.029233 | 5.2901 | 0.097799 |
| Bone Cancellous | 22.454 | 0.23423 | 18.478 | 0.82709 |

Phantom can be made to simulate the relative permittivity and conductivity listed in the above table. In some cases, phantom can be made to mimic tissue having the largest relative permittivity and conductivity (such as, muscle). Phantom can be made from a combination of diacetin and water. Various antennas along with the electronic circuitry, power source, housing, and implant can be positioned in the phantom (such as, submerged) to test the performance.

As confirmed by the table 1950, the substrate antenna 1200 exhibits the best return losses (S11) in the MICS and ISM bands. The PIFA antenna 1000 exhibits good return losses, but the amount of power radiated is less than that of the substrate antenna 1200. The amount of power radiated by the loop antenna 701 (and the helix antenna 1900) may be too low (particularly in the MICS band) to provide adequate performance. One of the reasons for such insufficient amount of power radiated by the loop antenna 701 (and the helix antenna 1900) may be space constraints for use with a hip implant (specifically, vertical constraints).

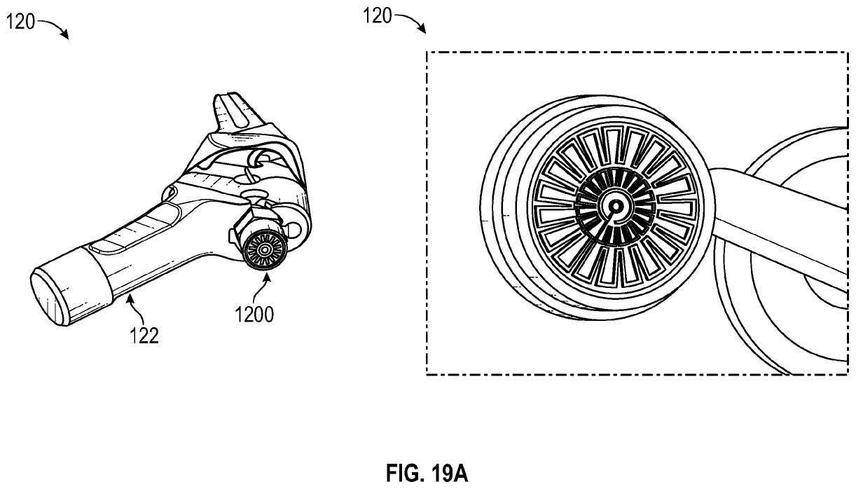

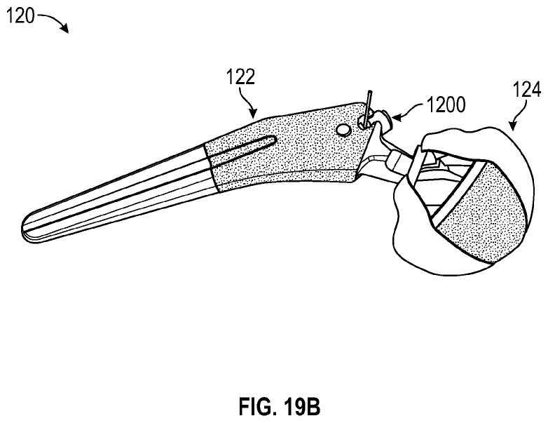

FIGS. 19A and 19B illustrate an example implementation of the femoral implant 120 with a sensor assembly that includes an implantable reporting processor having an antenna. The antenna can be a substrate antenna 1200 positioned in the cavity of the femoral stem 122, as shown in FIG. 19A. The substrate antenna 1200 can be positioned on a spacer, such as the spacer 1206 described herein. The femoral implant can include a femoral head 124 attached to the femoral stem 122 as shown in FIG. 19B.

As illustrated in FIGS. 19A and 19B, the antenna protrudes from the femoral implant 120. Any of the antennas disclosed herein can protrude from any of the implants.

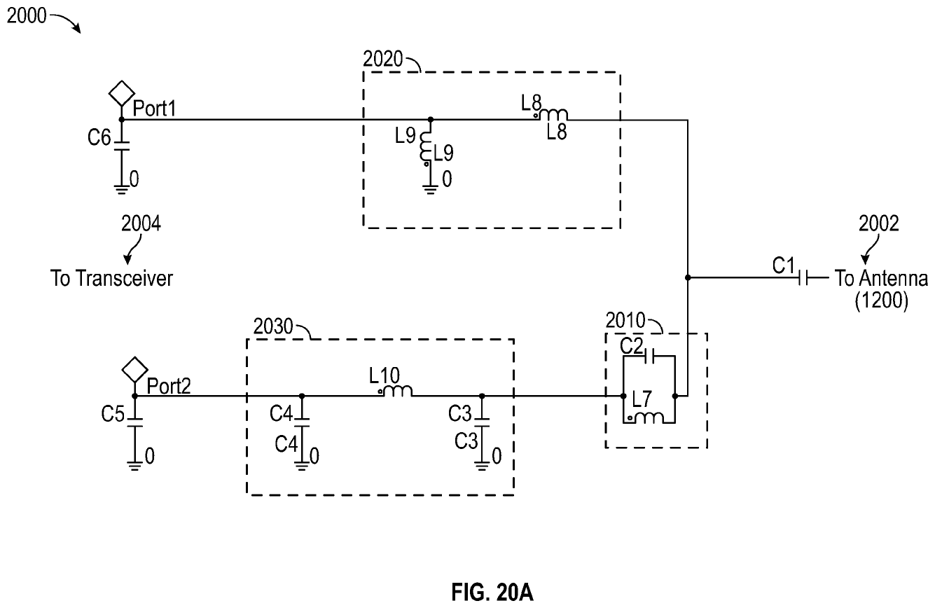

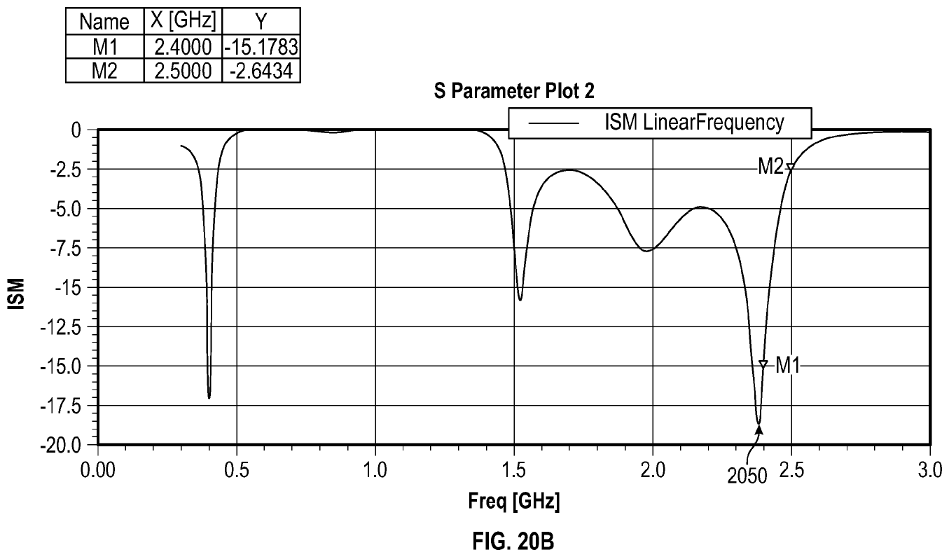

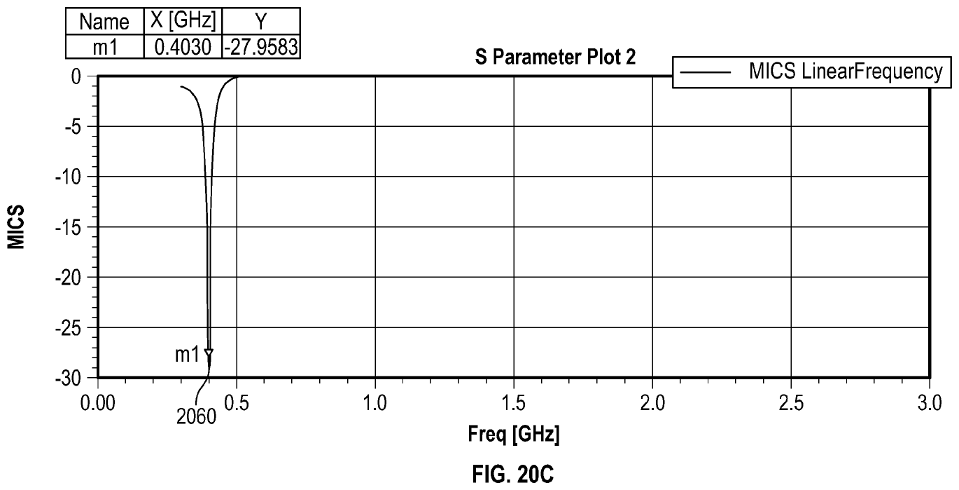

FIG. 20A illustrates a matching network circuitry 2000 (or matching circuitry) for the substrate antenna 1200, optimized for peak gain in the ISM and MICS frequency band. The substrate antenna 1200 can be tuned to improve performance in one or more frequency bands of interest. The matching circuitry 2000 can be similar to the matching circuitry 1150 for the PIFA antenna 1000 illustrated in FIG. 9E. Ports 2002 and 2004 can be similar to the ports 1160 and 1170 as described above.

Notch filter 2010 for processing signals in the first frequency band can be similar to the notch filter 1151 described above. L-network 2020 can process signals in the second frequency band similar to the matching network 1152 as described above. Additionally, Pi-network 2030 can process signals in the first frequency band similar to the matching network 1153.

FIG. 20B illustrates the return loss (or S11 parameter) of the substrate antenna 1200 connected to an optimized ISM band matching circuitry (such as, the matching circuitry 2000). As is shown, the substrate antenna 1200 resonance is almost in the center of the ISM band at 2.45 GHz (illustrated by 2050) with a peak return loss of about −18.75 dB. Comparing to FIG. 11B, the addition of an optimized ISM band matching circuitry (such as, the matching circuitry 2000) to the substrate antenna 1200 can improve the antenna performance (for instance, by shifting the resonance peak to the center of the ISM band).

FIG. 20C illustrates the return loss (or S11 parameter) of the substrate antenna 1200 connected to an optimized MICS band matching circuitry (such as, the matching circuitry 2000). As is shown, the substrate antenna 1200 return loss is almost in the center of the MICS band at 403.5 MHz (illustrated by 2060) with a peak return loss of about −29 dB. Comparing to FIG. 11B, the addition of an optimized ISM band matching circuitry (such as, the matching circuitry 2000) to the substrate antenna 1200 can improve the antenna performance (for instance, by shifting the resonance peak to the center of the MICS band and improving return loss).

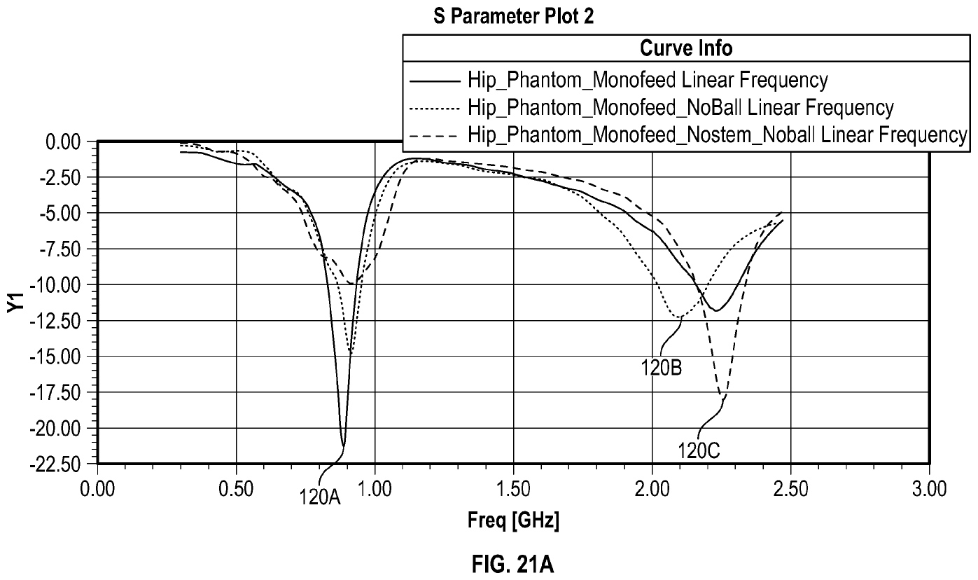

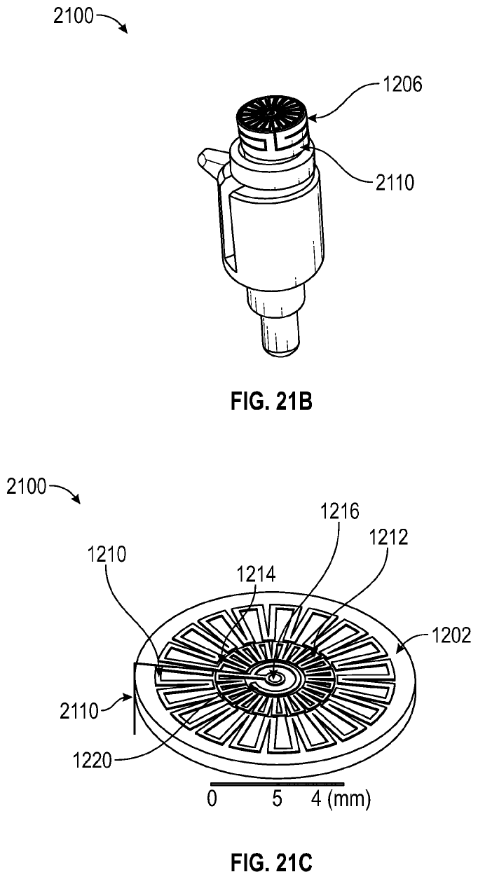

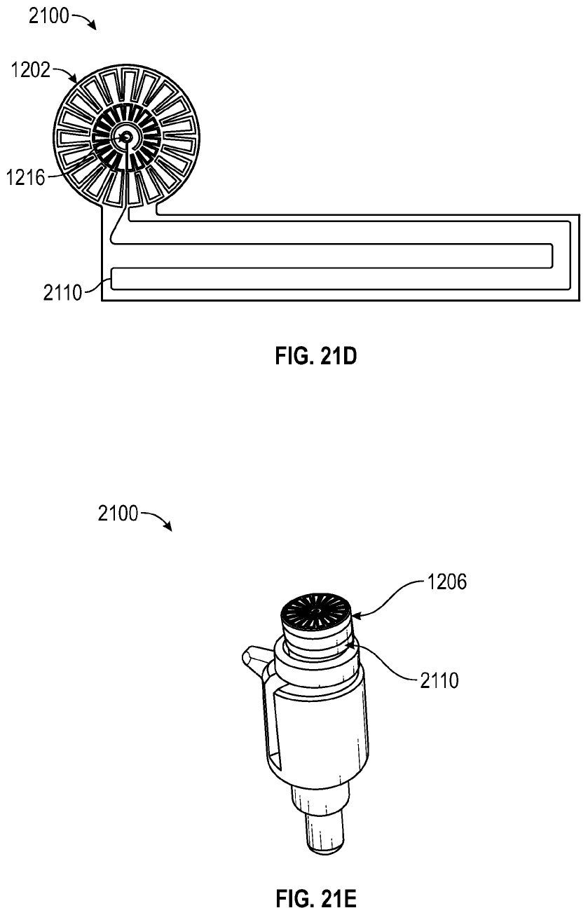

Different mechanical structures near the antenna can affect the antenna's resonance (and performance). FIG. 21A illustrates return loss plotted as a function of frequency for various configurations of the substrate antenna 1200. Plot 120C illustrates return loss of the substrate antenna 1200 tested without the femoral implant 120. Plot 120B illustrates return loss of the substrate antenna 1200 positioned within the cavity of the femoral stem 122 of the femoral implant 120, as shown in FIG. 19A. Plot 120A illustrates return loss of the substrate antenna 1200 within the cavity of femoral stem 122 and with the femoral head 124 attached to the femoral stem 122 of the femoral implant 120, as shown in FIG. 19B. FIG. 21A shows that as more conductive material of the femoral implant 120 (such as, metal) is positioned near the substrate antenna 1200, the lower and upper band responses of the substrate antenna 1200 shift to a higher peak return loss value particularly in the higher frequency band (lower return loss can indicate better resonance), thereby indicating a worsened performance of the antenna 1200 in the presence of the femoral implant 120.

In some cases, even though the antenna 1200 may be designed as an omnidirectional antenna in free space, performance of the antenna in presence of the femoral implant 120 may not be omnidirectional. In some cases, when the antenna 1200 is placed in the femoral stem 122, the presence of the femoral head 124 can create nulls in the three-dimensional radiation pattern of the antenna. These nulls may be in undesirable locations (or directions) when trying to transmit and received data in a direction out of the side of the patient. As a result, it may be advantageous to design the antenna to not have nulls at least in one or more locations (or directions) of interest.

The structure of the substrate antenna 1200 may be optimized to improve the antenna performance. For instance, antenna can be optimized so that there are no nulls in the one or more locations of interest. Optimization can involve adding more conductive material to the substrate antenna 1200 to counter the nulls (for instance, to shape the beam for peak gain in the one or more locations of interest). In some cases, this can involve wrapping a conductive material around the spacer 1206 of the substrate antenna 1200 (such substrate antenna can be referred to as a modified substrate antenna).

FIG. 21B is an example implementation of a modified substrate antenna 2100. The modified substrate antenna 2100 can be positioned on the spacer 1206 as described in connection with FIG. 10 and can include a conductive trace (or traces) 2110 wrapped around the spacer 1206. As illustrated in FIGS. 21B and 21D, the conductive trace 2110 can be doubled (to increase the electrical length of the modified substrate antenna 2100) and wrapped around opposite sides of the spacer 1206. The conductive trace 2110 can be positioned on spacer 1206 as illustrated with the goal of being located on the side opposite the femoral head 124 so as to shift the transmission nulls. In some cases, the conductive trace 2110 can be tripled, quadrupled, etc. or may not be folded at all. The conductive trace 2110 can be positioned (such as, printed, painted, or otherwise deposited) on the spacer 1206.

As shown in FIG. 21C, the conductive trace 2110 can be attached to one of the sections of the outer traces 1210 (or, in some implementations, one of the sections of the inner traces 1212 or to the feed point 1216) to form an electrical connection with the traces of the modified substrate antenna 2100. Such arrangement may be advantageous for deployment since the only required solder connection may be a connection at the feed point 1216.

The conductive trace 2110 can be wrapped around the base of the spacer 1206 one or more times. For instance, the conductive trace 2110 can be wrapped up and down the spacer 1206 to potentially cover additional surface area and further increase the electrical length of the modified substrate antenna 2100. In some cases, the conductive trace 2110 may be arranged such that the conductive trace 2110 is wrapped around a portion of the spacer 1206. As illustrated in FIG. 21E, the conductive trace 2110 may be wound in a helix pattern around the spacer 1206.

The conductive trace 2110 may include of a combination of two or more conductive traces of varying width. The conductive trace 2110 can include of a combination of two or more conductive traces wrapped fully or partially around the spacer.

Modified substrate antenna 2100 can include a stub (such as, the stub 1220), as illustrated in FIG. 21C. In some cases, the stub can be omitted.

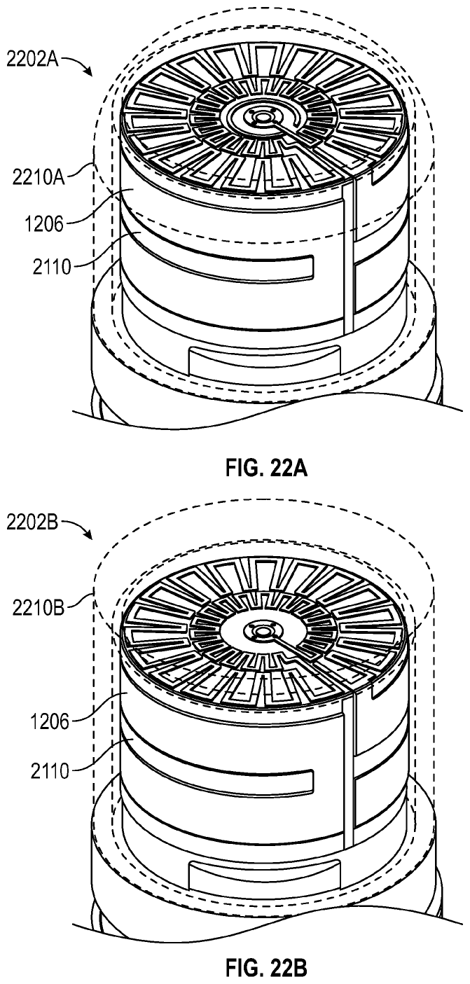

Any of the antennas disclosed herein can be covered with a cover, which can be similar to a radome. The cover can protect the antenna from being damaged and increase biocompatibility. This may be particularly applicable for antennas that protrude from an implant or are positioned in a different location from the implant. FIG. 22A illustrates a covered antenna 2202A. The illustrated antenna 2202A can be similar to the modified substrate antenna 2100. A cover 2210A can enclose the substrate antenna assembly, the conductive trace 2110, and the spacer 1206 supporting the conductive trace 2110, as illustrated. The cover 2210A (or any of the covers disclosed herein) can be made from a biocompatible material, such as PEEK or another biocompatible plastic. Using PEEK can be advantageous because of its resilience and shatter-proof properties. For antenna structures in which the spacer 1206 does not support conductive antenna components, a cover may cover only the antenna assembly without extending to the spacer 1206.

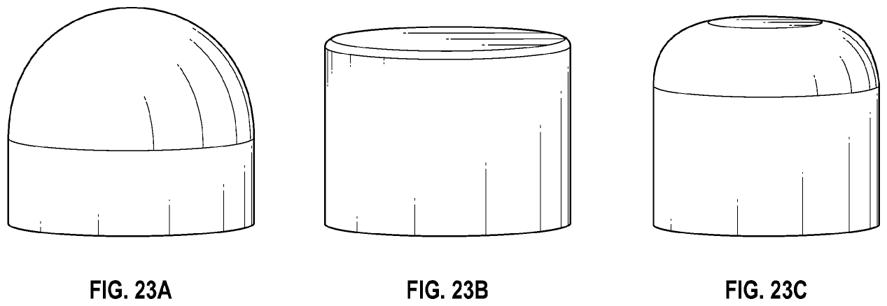

The cover 2210A has a spherical shape with a smooth top surface, as is also illustrated in FIG. 23A. Other cover shapes can be used. With reference to FIG. 22B, a covered antenna 2202B includes a cover 2210B that has a cylindrical shape, as is also illustrated in FIG. 23B. The illustrated antenna can be similar to the modified substrate antenna 2100 with the exception of not having a stub. In some cases, the stub can be included. The upper portion of the cover 2210B can be shorter than the upper portion of the cover 2210A, which is particularly visible from comparing FIG. 23A with FIG. 23B. FIG. 23C illustrates another cover shape that is spherical but has a flat top, and thus has a smaller height than the cover 2210A (compare FIG. 23A with FIG. 23C).

Different cover shapes can provide various advantages and disadvantages. Smoother cover shapes, such as that of the cover 2210A, can be preferable for being implanted into the body since it does not have sharp edges. However, smoother cover shapes may have a higher profile, which can make it more difficult for use in applications where vertical space is limited (such as, for hip implants). In some cases, the modified spherical cover shape illustrated in FIG. 23C may be preferable due to the combination of smoothness and low vertical profile.

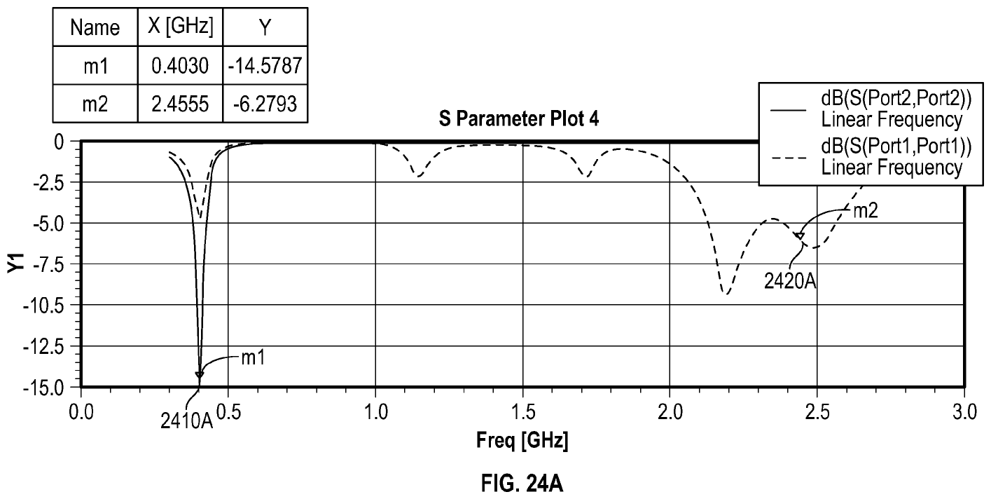

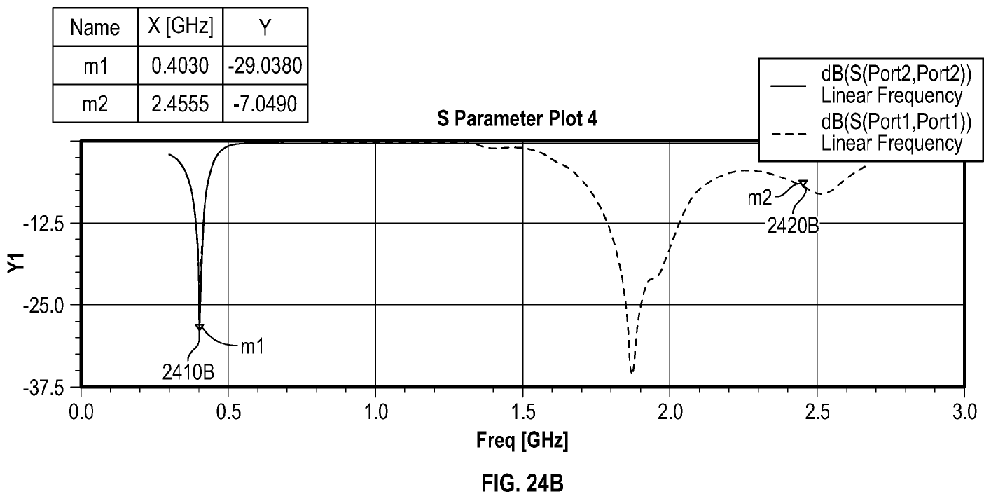

FIG. 24A illustrates a return loss (or S11 parameter) of the covered antenna 2202A as a function of frequency. The covered antenna 2202A can be connected to an optimized ISM and MICS band matching circuitry (such as, the matching circuitry 2000). As is shown, the covered antenna 2202A resonance is almost in the center of the ISM band at 2.4555 GHz (illustrated by 2420A) with a peak return loss of about −6.28 dB. The covered antenna 2202A return loss is almost in the center of the MICS band at 403.0 MHz (illustrated by 2410A) with a peak return loss of about −14.6 dB.

FIG. 24B illustrates a return loss (or S11 parameter) of the covered antenna 2202B as a function of frequency. The covered antenna 2202B can be connected to an optimized ISM and MICS band matching circuitry (such as, the matching circuitry 2000). As is shown, the covered antenna 2202B resonance is almost in the center of the ISM band at 2.4555 GHz (illustrated by 2420B) with a peak return loss of about −7.05 dB. The covered antenna 2202B return loss is almost in the center of the MICS band at 403.0 MHz (illustrated by 2410B) with a peak return loss of about −29.04 dB. While return losses of the covered antennas 2202A and 220B are acceptable, in simulation, the covered antenna 2202B exhibited better return loss than the covered antenna 2202A, likely due to the reduced vertical profile of the cover 2210B.

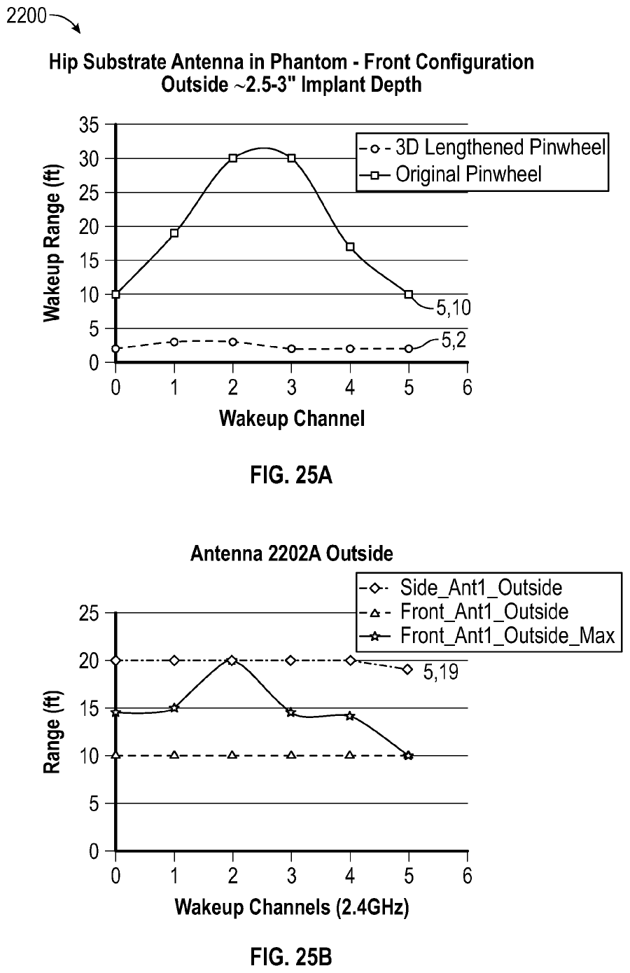

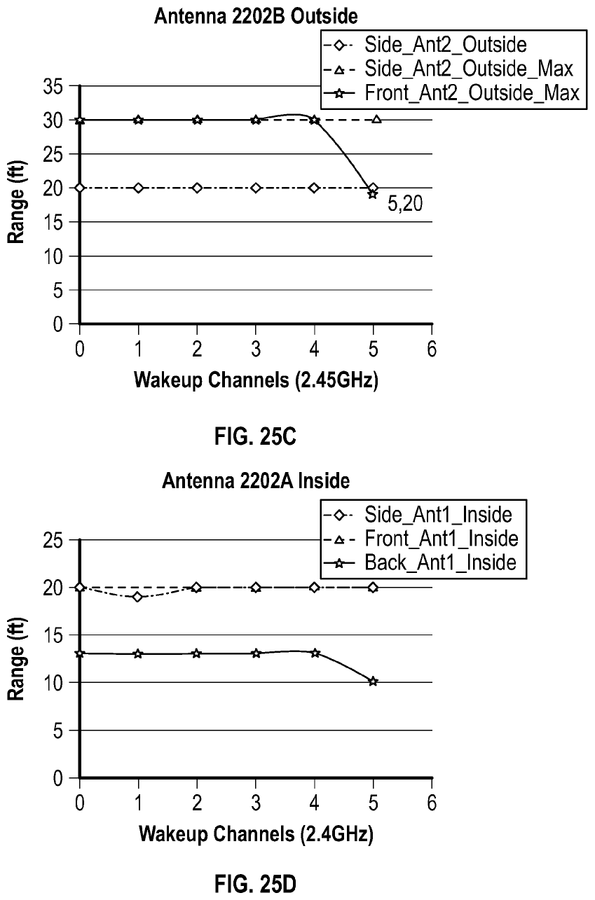

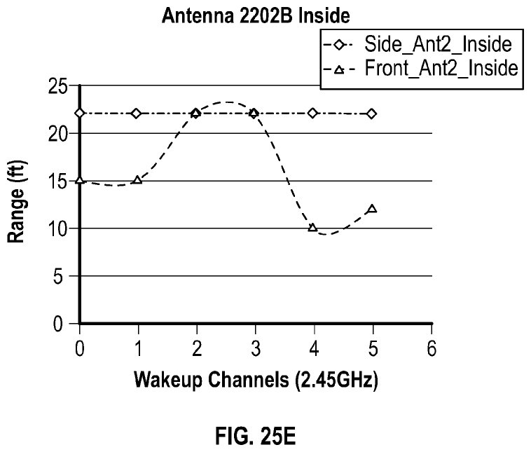

Communication range of various antenna configurations was tested in simulated conditions, in which an implant with the antenna has been submerged in a phantom to simulate positioning of the implant in the body. While the testing was performed in the MICS frequency band to determine the wake-up range, testing in the ISM frequency band would be performed similarly. FIG. 25A illustrates a wake-up range 2200 plotted as a function of different wake-up channels for various antenna configurations tested in freespace (such as, outdoors). The different wake-up channels can correspond to different frequencies in the MICS frequency band. In some cases, the substrate antenna 1200 and the modified substrate antenna 2100 are each placed in the femoral implant 120 (illustrated by FIG. 19B) and positioned in a phantom having a relative permittivity and conductivity similar to the muscle (as described above). The femoral head 124 of the femoral implant 120 can be turned 90 degrees from a base station, thereby centering the antenna in the phantom. The described, this configuration can represent a worst-case transmission scenario since the antenna is deep in the simulated muscle tissue and communication occurs out of the side of a patient lying in bed. The modified substrate antenna 2100 has an improved wake-up range across all wake-up channels in comparison to a substrate antenna 1200 when tested in such worst-case scenario. The wake-up communication range of the modified substrate antenna 2100 can exceed 30 feet and may not fall below 10 feet. As illustrated in FIG. 21B and others, wrapping a conductive trace 2110 around the spacer 1206 of the substrate antenna 1200 can improve the communication range of the antenna. In some cases, adding the conductive trace 2110 to the substrate antenna can resolve the problem of a transmission null in a direction of interest (such as, in the front direction).

Wake-up range of covered antennas 2202A and 2202B was similarly tested in freespace (such as, outdoors) as well as indoors. FIG. 25B illustrates wake-up range of the covered antenna 2202A tested outdoors as a function of different wake-up channels. Communication range of the antenna 2202A was tested in the side direction and the front direction (representing the worst case). In some cases, the wake-up communication range of the covered antenna 2202A when tested in freespace reaches 20 feet and does not fall below 10 feet. Other transmission directions were tested (such as, back), and it was confirmed that the wake-up communication range of the covered antenna 2202A in freespace did not fall below 10 feet.

FIG. 25C illustrates wake-up range of the covered antenna 2202B similarly tested outdoors as a function of different wake-up channels. As is illustrated, the wake-up communication range of the covered antenna 2202B in freespace reaches 30 feet and does not fall below 20 feet. In this test, the covered antenna 2202B exhibited longer wake-up communication range in the front configuration than the covered antenna 2202A. Other transmission directions were tested (such as, back), and it was confirmed that the wake-up communication range of the covered antenna 2202B in freespace did not fall below 20 feet.

Indoor testing was also performed, as illustrated in FIGS. 25D and 25E. Due to interference and distortion arising from multipath transmission, wake-up range would be expected to drop compared to freespace. Similarly to FIG. 25B, FIG. 25D illustrates wake-up range of the covered antenna 2202A tested indoors as a function of different wake-up channels. In some cases, the wake-up communication range of the covered antenna 2202A indoors reaches 20 feet and does not fall below 10 feet. Other transmission directions were tested (such as, back), and it was confirmed that the wake-up communication range of the covered antenna 2202A indoors did not fall below 10 feet.

Similarly to FIG. 25C, FIG. 25E illustrates wake-up range of the covered antenna 2202B tested indoors as a function of different wake-up channels. In some cases, the wake-up communication range of the covered antenna 2202B indoors reaches 20 feet and does not fall below 10 feet. Other transmission directions were tested (such as, back), and it was confirmed that the wake-up communication range of the covered antenna 2202B indoors did not fall below 10 feet.

### B. Sensor

As described herein, the sensor assembly 300 (or any of the other sensor assemblies disclosed herein) can include one or more sensors. “Sensor” refers to a device that can be utilized to do one or more of detect, measure and/or monitor: one or more different aspects of a body tissue (e.g., anatomy, physiology, metabolism, and function); one or more aspects of body or body segment condition or function (e.g., movement); and/or one or more aspects of the sensor assembly 300. The one or more sensors can be configured to detect movement (such as, steps), rotation, pressure, and the like that produce data associated with one or more physiological parameters of a patient or implant.

Representative examples of sensors suitable for use within the sensor assembly 300 include, for example, fluid pressure sensors, fluid volume sensors, contact sensors, position sensors, orientation sensors, pulse pressure sensors, blood volume sensors, blood flow sensors, chemistry sensors (e.g., for blood and/or other fluids), metabolic sensors (e.g., for blood and/or other fluids), impedance sensors, electrodes, accelerometers, gyroscopes, mechanical stress sensors and temperature sensors. Within some configurations, at least one sensor of the one or more sensors can have a Unique Sensor Identification number (“USI”), which specifically identifies the sensor.

The one or more sensors may be configured to detect, measure and/or monitor information relevant to the state of the sensor assembly 300 after implantation. The state of the sensor assembly 300 may include the integrity of the sensor assembly 300, the movement of the sensor assembly 300, the forces exerted on the sensor assembly 300 and other information relevant to the sensor assembly 300.

The one or more sensors may be configured to detect, measure and/or monitor information relevant body tissue (e.g., one or more physiological parameters of a patient) after implantation of the sensor assembly 300. Body tissue monitoring may include blood pressure, pH level, oxygen, carbon dioxide, potassium, iron, and/or glucose in the blood of the patient. The one or more sensors can include fluid pressure sensors, fluid volume sensors, pulse pressure sensors, blood volume sensors, blood flow sensors, chemistry sensors (e.g., for blood and/or other fluids), metabolic sensors (e.g., for blood and/or other fluids).

A radiopaque marker, or other type of marker, can be integrated with the one or more sensors. The radiopaque marker can track a location of the one or more sensors within the vasculature with standard fluoroscopy techniques.

### C. Processor/Controller

The sensor assembly 300 (or any of the other sensor assemblies disclosed herein) may include a processor (or controller) in electrical communication with the one or more sensors and/or the antenna(s) 312 (for instance, via a transmission and receive circuitry). The one or more sensors and the processor can be located on a printed circuit board. Alternatively, some or all of the one or more sensors may be located in or on another structure of the sensor assembly 300 separate from the printed circuit board. The processor, which can be any suitable microcontroller or microprocessor, can be configured to control the configuration and operation of one or more of the other components of the sensor assembly 300. For example, the processor can be configured to control the one or more sensors to sense relevant measurement data or physiological parameters, to store the measurement data generated by the one or more sensors in a memory, to generate messages, include the stored data as a payload, to packetize the messages, to provide the message packets to the antenna(s) 312 for transmission to a receiver (e.g., hub in the patient's body or a base station or other computing device outside the patient's body). The processor can be configured to execute commands received from a base station or other computing device via the antenna(s) 312. For example, the processor can be configured to receive configuration data from the base station, and to provide the configuration data to the component of the sensor assembly 300 to which the base station directed the configuration data. If the base station directed the configuration data to the processor, then the processor can configure itself in response to the configuration data.

The processor can cause the one or more sensors to measure, to detect, to determine if a measurement is a qualified or valid measurement, to store the data representative of a valid measurement, and to cause the antenna(s) 312 to transmit the stored data to a base station or other source external to the sensor assembly 300. In response to being polled by a base station or by another device external to the sensor assembly 300, the processor can generate conventional messages having payloads and headers. The payload scan can include the stored samples of the signals that the one or more sensors generated. The headers can include the sample partitions in the payload, a time stamp indicating the time at which the sensor acquired the samples, an identifier (e.g., serial number) of the sensor assembly 300, and/or a patient identifier (e.g., a number or name).

The processor can generate data packets that include the messages according to a conventional data-packetizing protocol. Each packet can also include a packet header that includes, for example, a sequence number of the packet so that the receiving device can order the packets properly even if the packets are transmitted or received out of order. The processor can encrypt some or all parts of each of the data packets, for example, according to a conventional encryption algorithm, and error encodes the encrypted data packets. For example, the processor can encrypt at least the sensor assembly 300 and patient identifiers to render the data packets compliant with the Health Insurance Portability and Accountability Act (“HIPAA”). The processor can provide the encrypted and error-encoded data packets to the antenna(s) 312, which, via the filter, transmits the data packets to a destination, such as the base station 4 (shown in FIG. 1) or a receiver external to the sensor system. The antenna(s) 312 can transmit the data packets according to any suitable data-packet-transmission protocol.

Alternate configurations of the sensor assembly 300. For example, the antenna(s) 312 can perform encryption or error encoding instead of, or complementary to, the processor. Furthermore, the sensor assembly 300 can include components other than those described herein and can omit one or more of the components described herein.

The sensor assembly 300 may include a memory circuit (not shown) that can be any suitable nonvolatile memory circuit, such as EEPROM or FLASH memory. The memory can be in electrical communication with the processor, the antenna(s) 312, and/or the one or more sensors. The memory can be configured to store data written, for example, by the processor or the antenna(s) 312, and to provide data in response to a read command from the processor.

### D. Power Source

The sensor assembly 300 (or any of the other sensor assemblies disclosed herein) can include one or more power sources. For example, the sensor assembly can include one or more batteries and/or supercapacitors. The power source may be sized to fit within the body part (such as, the femur) with the remainder of the sensor assembly 300. In other configurations, the sensor assembly 100a, 100b may be powered by a power source at a remote location from the body part, either in the patient's body or outside the patient's body.

The power source can be any suitable battery, such as a Lithium Carbon Monofluoride (LiCFx) battery or solid state battery, or other storage cell capable of storing energy (e.g., a supercapacitor) for powering the processor for an expected lifetime of the sensor assembly 300 (e.g., at least one month or at least six months). The power source may receive sufficient energy from the sensor reaction by-product to maintain a minimal power capacity for sustaining micro-controller memories, real time clocks and/or SRAMs sleep modes.

Replacing a power source implanted in a patient is often not desirable at least because it involves an invasive procedure that can be relatively expensive and that can have adverse side effects, such as infection and soreness. Thus, the power source may be rechargeable. For example, the power source may be recharged using integrated circuitry on an ASIC chip. As another example, the battery may be charged inductively. For example, any of the sensor assemblies disclosed herein can include an antenna for wireless charging (such as, a coil antenna). Any of garments disclosed herein can include a wireless power transmitter to facilitate inductive charging.

### E. Spinal Implant

As described herein, any of the sensor assemblies disclosed herein can be implanted in any joint or body part. For example, any of the sensor assemblies described herein can be implanted in the spine. Spinal procedures such as spinal fusion can generally refer to the surgery and associated implantable medical devices such as a spinal implant system (for instance, a spinal fusion implant such as a spinal interbody cage or spacer, rod or plate, or a spinal non-fusion implant such as an artificial disc or expandable rod). Examples of spinal devices and implants include pedicle screws, spinal rods, spinal wires, spinal plates, spinal cages, artificial discs, facet implants, bone cement, as well as combinations of these (e.g., one or more pedicle screws and spinal rods, one or more pedicle screws and a spinal plate). In addition, medical delivery devices for the placement of spinal devices and implants, along with one or more sensors, may also be an intelligent medical device according to the present disclosure. Examples of medical delivery devices for spinal implants include kyphoplasty balloons, catheters (including thermal catheters and bone tunnel catheters), bone cement injection devices, microdiscectomy tools and other surgical tools.

An intelligent implant (or sensor assembly) can include an implantable reporting processor (IRP) incorporated into a spinal cage or an interbody spacer used during spinal fusion. The spinal cage can be inserted along any point of the patient's spine. For example, the spinal cage can be inserted to provide for spinal fusion in the lumbar spine, thoracic spine, and/or cervical spine. The spinal cage can be configured to take the load from adjacent vertebrae and can include an opening that can receive an (IRP). In examples where a spinal cage and separate IRP cartridge is used, the physician will couple the IRP with the spinal cage to form the intelligent implant. In alternative configurations, the IRP may be integrated with the implant and contain any of the features of the cartridge IRP described herein. The physician can then fill the spinal cage with material to help retain the IRP and provide for enhanced fusion between the adjacent vertebrae. For example, the spinal cage can be filled with body material (e.g., bloody sawdust), other biological material, or other synthetics. As will be discussed in more detail below, the IRP can form a cartridge that can be permanently or reversibly insertable into the spinal cage. The cartridge can include an antenna, an inertial measurement unit (IMU), and/or additional sensors that can sense and track a patient's movements.

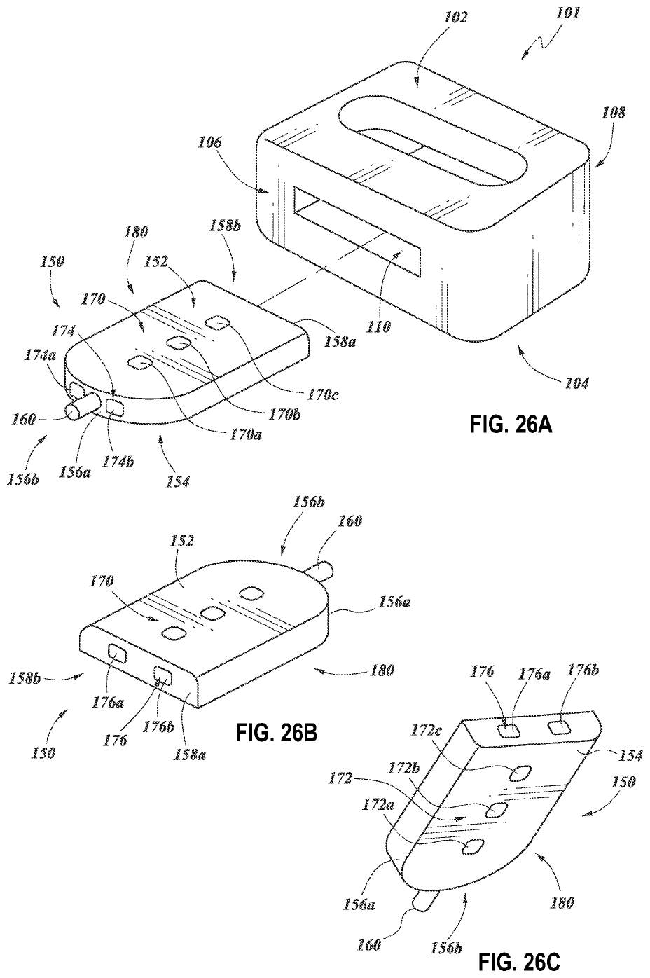

FIG. 26A illustrates a perspective view of an intelligent implant 101 in the form of a spinal cage that includes an implantable reporting processor 150. The implantable reporting processor 150 can interface with a spinal cage/interbody spacer like that shown in FIG. 26A. For example, the components can be assembled intraoperatively. The spinal cage can come in a variety of different shapes and sizes. As illustrated, the spinal cage can comprise a top surface 102, a bottom surface 104, a medial surface 106, and lateral surface 108. The medial surface 106 can include an opening 110 and/or the lateral surface 108 can include an opening 112. The opening 110 and/or the opening 112 can allow the implantable reporting processor 150 to be inserted and secured within the spinal cage of the intelligent implant 101.

The implantable reporting processor 150 can include a housing 180 that encloses a battery, an electronics assembly, an antenna, and/or one or more sensors. The one or more sensors can include any of the sensors described herein. The housing 180 can include a cover or a casing that encases and secures the various components of the implantable reporting processor 150. For example, as illustrated in FIG. 26A, the implantable reporting processor 150 is in the form of a cartridge for insertion into the patient. The cartridge can be any shape or size such that it can be inserted into the spinal cage. For example, the implantable reporting processor 150 of FIG. 26A is thin and wafer shaped to allow it to be inserted through the opening 110 or the opening 112 of the spinal cage. The implantable reporting processor 150 can be generally D-shaped with a generally curved medial end 156b and a generally straight lateral end 158b. However, the implantable reporting processor 150 can be cylindrical or any other shape or size. As shown in FIGS. 26B-26C, the implantable reporting processor 150 can include a housing 180 that covers and encloses the battery and the electronics assembly. In some embodiments, the implantable reporting processor 150 can include a top surface 152, a bottom surface 154, a medial surface 156a, and a lateral surface 158a. The implantable reporting processor 150 can include an antenna 160, which can be any of the antennas disclosed herein, such as the antenna 312, 701, 1000, 1200, 1700, 1800, 1900, or 2100. In some embodiments, the antenna 160 of the implantable reporting processor 150 can have a radome extending from the exterior profile of the implantable reporting processor 150, for example the medial end 156b of the implantable reporting processor 150. When assembled with the spinal cage/interbody spacer, the radome can protrude from the exterior profile of the spacer. This arrangement allows for communication even if the implantable reporting processor 150 and/or spacer has a metallic material. As described in more detail here, the implantable reporting processor 150 and/or spinal cage/interbody spacer can communicate with external devices, for example using Bluetooth Low Energy, to transmit collected data or receive programming and configuration data. The antenna 160 can be optimized for frequencies in the ISM and MICS bands.

As will be discussed in more detail below, the implantable reporting processor 150 can include one or more sensors positioned on the various surfaces of the implantable reporting processor 150. As shown in FIGS. 26A-26C, the implantable reporting processor 150 can include at least one sensor 170. The at least one sensor 170 can be hermetically sealed and mounted inside or attached to a surface of the implantable reporting processor 150, for example on the top surface 152 of the implantable reporting processor 150. As illustrated in FIGS. 26A-26C, at least one sensor 170 of the implantable reporting processor 150 can include a sensor 170a, sensor 170b, and sensor 170c. The sensors 170a, 170b, 170c can be positioned in series.

The at least one sensor 170 can include strain or force sensors to detect strain or force on the surface of the implantable reporting processor 150 as means to detect fusion between two adjacent vertebrae in which the spacer is positioned because load increases as fusion progresses.

The at least one sensor 170 can include a vibration sensor detect acoustic emissions associated with scratching/abrasion of the interbody spacer against adjacent vertebrae. The extent of acoustic emissions changes (most likely decreasing) as intervertebral fusion progresses.

The vibration sensor can be combined with accelerometer(s) to collect acoustic emission (vibration) measurements when the patient is in known activities such as walking. The accelerometer can be low power AC or DC accelerometer. The accelerometer(s) can be hermetically sealed within the implantable reporting processor 150. The accelerometer(s) can measure the inclination angle of the spine relative to gravity vector; spine angle relative to the gravity vector provides a center of mass measurement; the center of mass measurement correlates to the recovery, pain level, and/or health status of the patient. The accelerometer(s) can measure activity patterns of the patient (such as walking) and trigger collection of data by the accelerometer(s) to occur during targeted activities such as walking.

Although the at least one sensor 170 is illustrated on the top surface of the implantable reporting processor 150, sensor(s) may be positioned on any other surface of the implantable reporting processor 150 as described below. Moreover, in other embodiments, the at least one sensor 170 may be incorporated into the spacer itself.

Additionally or alternatively, the implantable reporting processor 150 can include at least one sensor 172 on the bottom surface 154 of the implantable reporting processor 150. As illustrated in FIGS. 26A-26C, at least one sensor 172 of the implantable reporting processor 150 can include a sensor 172a, sensor 172b, and sensor 172c. The sensors 172a, 172b, 172c can be positioned in series.

In some configurations, the implantable reporting processor 150 can include at least one sensor 174 on the medial surface 156a of the implantable reporting processor 150. As illustrated in FIGS. 26A-26C, at least one sensor 174 of the implantable reporting processor 150 comprises a sensor 174a and a sensor 174b. However, at least one sensor 174 can include a greater or fewer number of sensors in various configurations. In some configurations, the implantable reporting processor 150 can include at least one sensor 176 on the lateral surface 158a of the implantable reporting processor 150. As illustrated in FIGS. 26A-26C, at least one sensor 176 of the implantable reporting processor 150 can comprises a sensor 176a and a sensor 176b. However, at least one sensor 176 can include a greater or fewer number of sensors in various configurations. In some embodiments, at least one sensor 170, at least one sensor 172, at least one sensor 174, and at least one sensor 176 can be embedded within the material of the housing 180.

The implantable reporting processor 150 can be in the form of a cartridge that can be inserted into the spinal cage through either the opening 110 of the medial surface 106 or the opening 112 of the lateral surface 108. In some embodiments, the implantable reporting processor 150 can form a positive connection with the spinal cage before the spinal cage is inserted into the patient. For example, the positive connection can be any of a number of mechanical connections such as snap fits, locks, twists, mated threads, etc. In some embodiments, the implantable reporting processor 150 can be inserted into the spinal cage reversibly. The reversible connection between the implantable reporting processor 150 and the spinal cage can provide the physician with the option of removing the cartridge in situations where any maintenance may need to be performed on the implantable reporting processor 150 once it is inserted into the patient. A reversible connection can allow the implantable reporting processor 150 to be removed in the instance when a battery may need to be replaced.

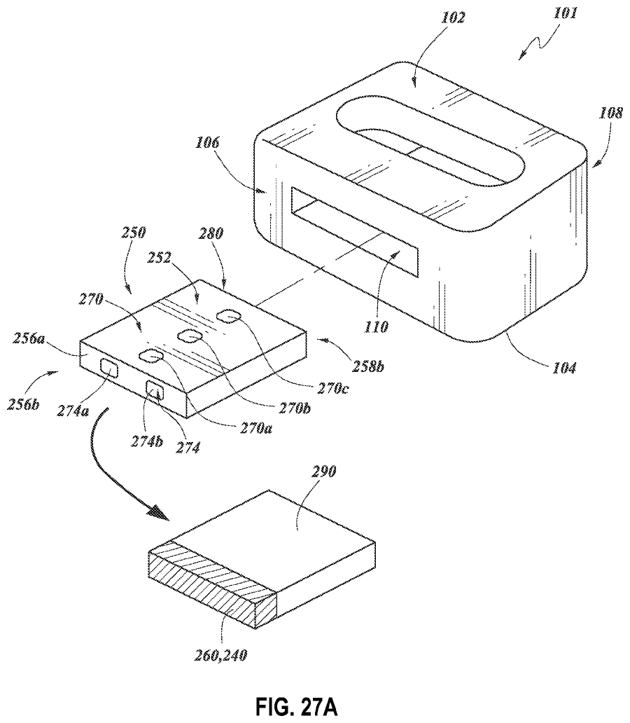

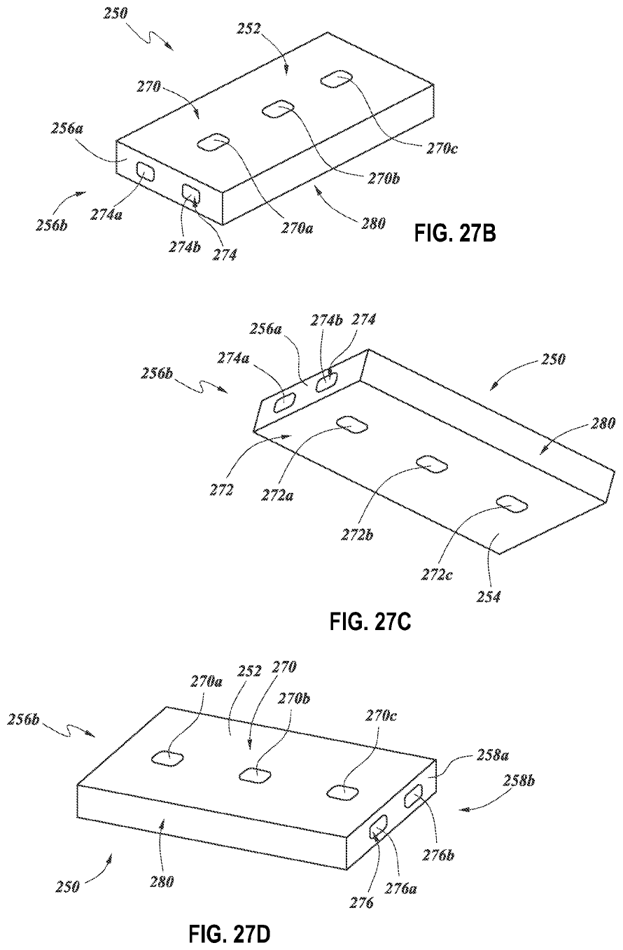

FIG. 27A illustrates the intelligent implant 101 that includes an implantable reporting processor 250 for insertion into the spinal cage of the intelligent implant 101. The implantable reporting processor 250 can include any of the features described above with respect to implantable reporting processor 150. FIG. 27A illustrates an implantable reporting processor 250 for insertion into the spinal cage that includes an antenna 260 that does not extend from a surface of the implantable reporting processor 250. The antenna 260 can be any of the antennas disclosed herein, such as the antenna 160, 312, 701, 1000, 1200, 1700, 1800, 1900, or 2100

The implantable reporting processor 250, like the implantable reporting processor 150, can include a housing 280 that encloses a battery 290, an electronics assembly 240, the antenna 260, and a plurality of sensors. Similar to the implantable reporting processor 150, the housing 280 of the implantable reporting processor 250 comprises a cover or a casing that encases and secures the various components of the implantable reporting processor 250. For example, as illustrated in FIG. 27A, the implantable reporting processor 250 is in the form of a cartridge for insertion into the patient. The implantable reporting processor 250 can be any shape or size such that it can be inserted into FIGS. 27A-27C is thin and wafer shaped to allow it to be inserted through the opening 110 or the opening 112 of the spinal cage. However, as discussed above with regard to the implantable reporting processor 150, the implantable reporting processor 250 can be cylindrical or any other shape or size. As shown in FIGS. 27A-27C, the implantable reporting processor 250 can include a top surface 252, a bottom surface 254, a medial surface 256a and a lateral surface 258a. The implantable reporting processor 250 can include the housing 280 that covers and encloses the antenna 260 and the electronics assembly 240 at a medial end 256b and the battery 290 at a lateral end 258b of the implantable reporting processor 250.

Similar to the implantable reporting processor 150, the implantable reporting processor 250 can include a plurality of sensors (e.g., ultrasound sensors) positioned on the various surfaces of the implantable reporting processor 250. As shown in FIGS. 27A-27D, the implantable reporting processor 250 can include at least one sensor 270 on the top surface 252 of the implantable reporting processor 250. As illustrated in FIGS. 27A-27C, at least one sensor 270 of the implantable reporting processor 250 are made up of a sensor 270a, a sensor 270b, and a sensor 270c. The sensors 270a, 270b, 270c can be positioned in series along the top surface 252 of the implantable reporting processor 250.

Additionally or alternatively, the implantable reporting processor 250 can include at least one sensor 272 on the bottom surface 254 of the implantable reporting processor 250. As illustrated in FIGS. 27A-27C, at least one sensor 272 can include a sensor 272a, a sensor 272b, and a sensor 272c. The sensors 272a, 272b, 272c can be positioned in series along the bottom surface 254 of the implantable reporting processor 250.

In some configurations, the implantable reporting processor 250 can include at least one sensor 274 on the medial surface 256a of the implantable reporting processor 250. As illustrated in FIGS. 27A-27C, at least one sensor 274 of the implantable reporting processor 250 are made up of a sensor 274a and a sensor 274b. However, at least one sensor 274 can include a greater or fewer number of sensors in any number of arrangements. In some configurations, the implantable reporting processor 250 can include at least one sensor 276 on the lateral surface 258a of the implantable reporting processor 250. As illustrated in FIGS. 27A-27C, at least one sensor 276 of the implantable reporting processor 250 can comprise a sensor 276a and a sensor 276b. However, at least one sensor 276 can include a great or fewer number of sensors in a variety of configurations. In some embodiments, at least one sensor 270, at least one sensor 272, at least one sensor 274, and at least one sensor 276 can be embedded within the material of the housing 280.

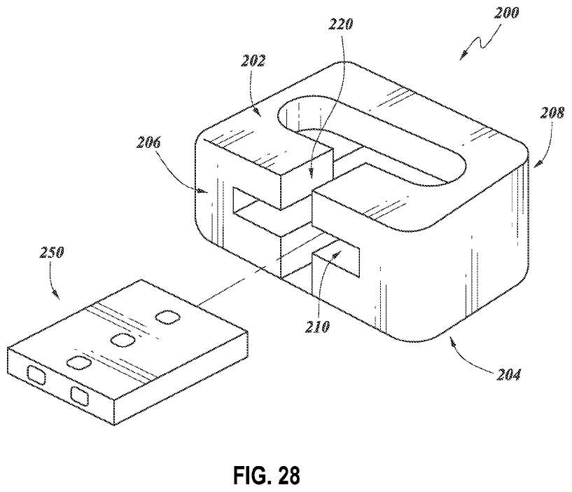

FIG. 28 illustrates an intelligent implant 200 comprising a spinal cage. The intelligent implant 200 can include any of the features described above with respect to intelligent implant 101. The intelligent implant 200 can include a top surface 202, a bottom surface 204, a medial surface 206, and a lateral surface 208. The intelligent implant 200 includes an opening 210 in the window 220 and an opening 212 in the lateral surface 208. The intelligent implant 200 further includes a window 220 positioned in either the medial end or the lateral end of the intelligent implant 200. In embodiments where the implantable reporting processor includes an antenna that does not extend out of the intelligent implant 200, the window 220 can allow for signals to be transmitted out of the intelligent implant 200 without any interference. This is particularly the case when the housing 280 of the implantable reporting processor 250 comprises a material such as metal. In embodiments where the housing 280 comprises material such as PEEK, signal transmission is not blocked and therefore an intelligent implant 101 without a window can be used.

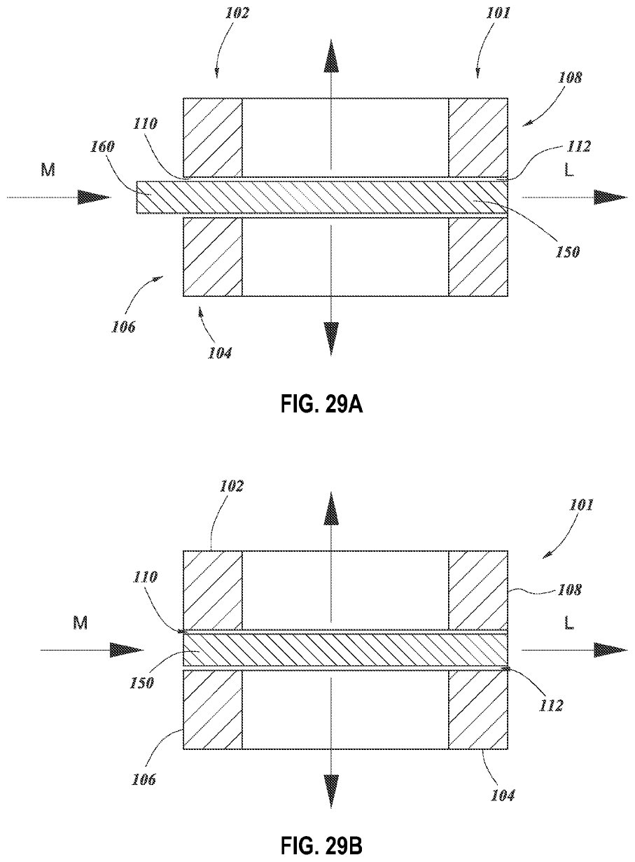

FIGS. 29A-29B illustrate cross-sectional medial-lateral views of the intelligent implant 101 with either the implantable reporting processor 150 or the implantable reporting processor 250 inserted into the spinal cage. FIG. 29A illustrates a cross-sectional view wherein the antenna 160 extends out of the opening 110 of the medial surface 106. FIG. 29B illustrates a cross-sectional view wherein the entirety of the implantable reporting processor 250 does not extend from either end of the intelligent implant 101 and is contained between the medial surface 106 and the lateral surface 108 of the intelligent implant 101.

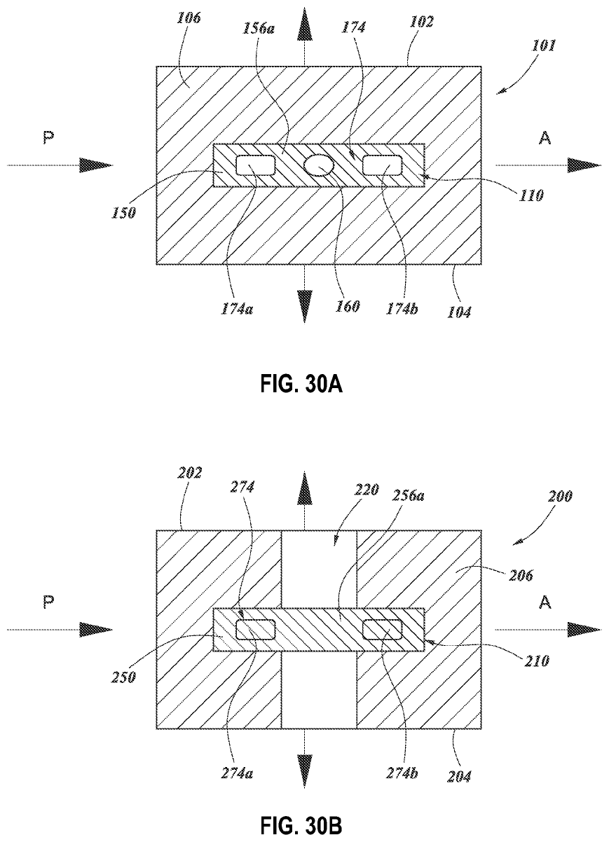

FIGS. 30A-30B illustrate proximal-anterior views of either the implantable reporting processor 150 or the implantable reporting processor 250 inserted into the intelligent implant 101 or the intelligent implant 200. In FIG. 29B, the window 220 of the implantable reporting processor 250 allows a top and/or bottom portion of the antenna 260 on the medial end 256b of the implantable reporting processor 250 to be exposed. As this is where the antenna 260 is positioned under the housing 280, the antenna 260 can transmit signals out of the intelligent implant 200.

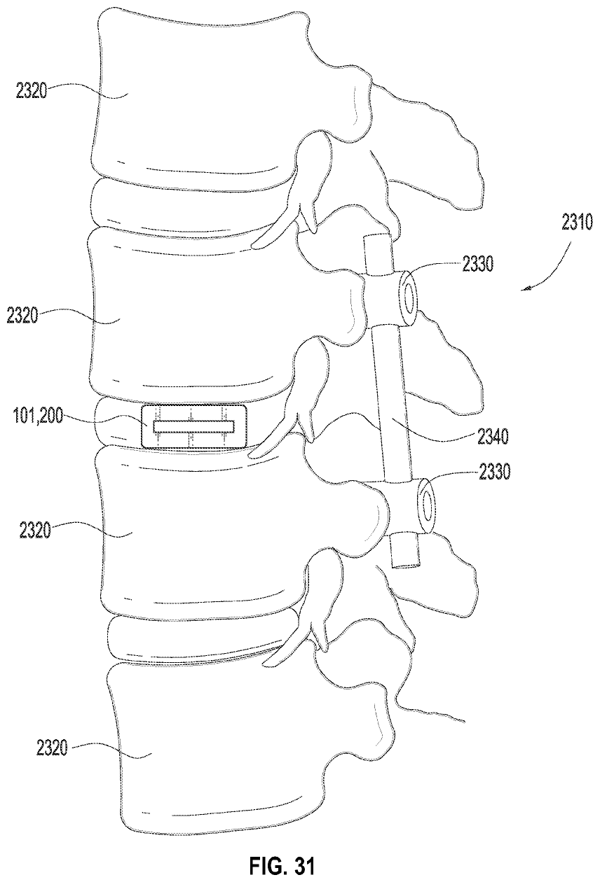

FIG. 31 illustrates a posterior-anterior view of the intelligent implant 101, 200 inserted into the spine 2310 of a patient during spinal fusion. The intelligent implant 101, 200 can be in the form of a spinal cage and is inserted between adjacent vertebrae 2320. The adjacent vertebrae 2320 can include a plurality of pedicle screws 2330 and rod 2340 that help to secure the vertebrae 2320 in place during and after spinal fusion.

Examples of spinal implant sensor assemblies in which the intelligent implants disclosed herein may be incorporated, are described in U.S. Patent Application No. 63/378,588, filed on Oct. 6, 2022, the disclosure of which is incorporated herein in its entirety.

### III. Additional Embodiments and Terminology

All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Such documents may be incorporated by reference for the purpose of describing and disclosing, for example, materials and methodologies described in the publications, which might be used in connection with the present disclosure. The publications discussed above and throughout the text are provided solely for their disclosure prior to the filing date of the present application. Nothing herein is to be construed as an admission that the inventors are not entitled to antedate any referenced publication by virtue of prior invention.

Although certain systems and methods have been described herein with respect to a hip implant, the systems and methods described herein can be applied to any joint implant or any implant in general. Systems and methods described herein are not limited to joints and are generally applicable to body parts (such as, the spine). Although certain embodiments and examples have been described herein, it will be understood by those skilled in the art that many aspects of the sensor assemblies shown and described in the present disclosure may be differently combined and/or modified to form still further embodiments or acceptable examples. All such modifications and variations are intended to be included herein within the scope of this disclosure. A wide variety of designs and approaches are possible. No feature, structure, or step disclosed herein is essential or indispensable.

For purposes of this disclosure, certain aspects, advantages, and novel features are described herein. It is to be understood that not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Moreover, while illustrative embodiments have been described herein, the scope of any and all embodiments having equivalent elements, modifications, omissions, combinations (e.g., of aspects across various embodiments), adaptations and/or alterations as would be appreciated by those in the art based on the present disclosure. The limitations in the claims are to be interpreted broadly based on the language employed in the claims and not limited to the examples described in the present specification or during the prosecution of the application, which examples are to be construed as non-exclusive. Further, the actions of the disclosed processes and methods may be modified in any manner, including by reordering actions and/or inserting additional actions and/or deleting actions. It is intended, therefore, that the specification and examples be considered as illustrative only, with a true scope and spirit being indicated by the claims and their full scope of equivalents.

The terms “approximately,” “about,” and “substantially” as used herein represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount.

Conditional language used herein, such as, among others, “can,” “might,” “may,” “e.g.,” and the like, unless specifically stated otherwise, or otherwise understood within the context as used, is generally intended to convey that some embodiments include, while other embodiments do not include, certain features, elements, and/or states. Thus, such conditional language is not generally intended to imply that features, elements, blocks, and/or states are in any way required for one or more embodiments or that one or more embodiments necessarily include logic for deciding, with or without author input or prompting, whether these features, elements and/or states are included or are to be performed in any particular embodiment.

The methods disclosed herein may include certain actions taken by a clinician; however, the methods can also include any third-party instruction of those actions, either expressly or by implication. For example, actions such as “releasing the sensor assembly” include “instructing release of the sensor assembly.”

The various illustrative logical blocks, modules, routines, and algorithm steps described in connection with the embodiments disclosed herein can be implemented as electronic hardware, computer software, or combinations of both. To clearly illustrate this interchangeability of hardware and software, various illustrative components, blocks, modules, and steps have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

Moreover, the various illustrative logical blocks and modules described in connection with the embodiments disclosed herein can be implemented or performed by a machine, such as a general purpose processor device, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor device can be a microprocessor, but in the alternative, the processor device can be a controller, microcontroller, or state machine, combinations of the same, or the like. A processor device can include electrical circuitry configured to process computer-executable instructions. In another embodiment, a processor device includes an FPGA or other programmable device that performs logic operations without processing computer-executable instructions. A processor device can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor device may also include primarily analog components. For example, some or all of the signal processing algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, routine, or algorithm described in connection with the embodiments disclosed herein can be embodied directly in hardware, in a software module executed by a processor device, or in a combination of the two. A software module can reside in RAM memory, flash memory, ROM memory, EPROM memory, EEPROM memory, registers, hard disk, a removable disk, a CD-ROM, or any other form of a non-transitory computer-readable storage medium. An example storage medium can be coupled to the processor device such that the processor device can read information from, and write information to, the storage medium. In the alternative, the storage medium can be integral to the processor device. The processor device and the storage medium can reside in an ASIC. The ASIC can reside in a user terminal. In the alternative, the processor device and the storage medium can reside as discrete components in a user terminal.

### IV. Example Implementations

Implementation 1: An implantable system for a body part of a patient, the system comprising:
an electronic circuitry comprising:
at least one sensor;
a processor configured to receive data obtained by the at least one sensor; and
a communication circuitry connected to the processor and configured to transmit the data obtained by the at least one sensor; and
an antenna comprising a plurality of conductive traces present on a substrate, the antenna connected to the communication circuitry and further configured to facilitate transmission of the data.

Implementation 2: The implantable system of implementation 1, wherein the plurality of conductive traces are painted or printed on the substrate.

Implementation 3: The implantable system of any of implementations 1 to 2, wherein the substrate comprises at least one of liquid crystal polymer, polyimide, or polyamide.

Implementation 4: The implantable system of any of implementations 1 to 3, wherein the plurality of conductive traces comprises a first plurality of conductive traces surrounding a second plurality of conductive traces, the first plurality of conductive traces connected to the second plurality of conductive traces.

Implementation 5: The implantable system of implementation 4, wherein the first plurality of conductive traces is arranged in a first ring and the second plurality of conductive traces is arranged in a second ring, and wherein the first ring surrounds the second ring.

Implementation 6: The implantable system of implementation 5, wherein the substrate is circular.

Implementation 7: The implantable system of any of implementations 4 to 6, wherein the first plurality of conductive traces comprises a first plurality of interconnected petals and the second plurality of conductive traces comprises a second plurality of interconnected petals.

Implementation 8: The implantable system of implementation 7, wherein distance between petals in at least one of the first or second plurality of interconnected petals affects resonance of the antenna.

Implementation 9: The implantable system of any of implementations 7 to 8, wherein at least some petals in at least one of the first or second plurality of interconnected petals have rounded corners, thereby improving resonance of the antenna in one or more frequency bands of interest.

Implementation 10: The implantable system of any of implementations 7 to 9, wherein the plurality of conductive traces further comprises a stub connected to the second plurality of conductive traces.

Implementation 11: The implantable system of implementation 10, wherein the stub is curved.

Implementation 12: The implantable system of any of implementation 10 to 11, wherein the antenna is configured to transmit and receive in a frequency band, and wherein a length of the stub is selected to facilitate transmission and reception in the frequency band.

Implementation 13: The implantable system of any of implementations 1 to 12, wherein the substrate is supported by a spacer separating the antenna from the electronic circuitry.

Implementation 14: The implantable system of implementation 13, wherein the antenna further comprises an additional conductive trace supported by the spacer and connected to a conductive trace of the plurality of conductive traces.

Implementation 15: The implantable system of implementation 14, wherein the additional conductive trace is at least partially wrapped around the spacer.

Implementation 16: The implantable system of implementation 15, wherein the additional conductive trace is arranged into two sections that at least partially wrapped around opposite sides of the spacer.

Implementation 17: The implantable system of any of implementations 13 to 16, wherein the antenna is configured to transmit and receive in a frequency band, and wherein a height of the spacer is selected to facilitate transmission and reception in the frequency band.

Implementation 18: The implantable system of any of implementations 13 to 17, further comprising a cover enclosing the antenna and the spacer.

Implementation 19: The implantable system of implementation 18, wherein the cover is spherical, cylindrical, or spherical with a flat top.

Implementation 20: The implantable system of any of implementations 18 to 19, wherein the cover is made out of biocompatible material.

Implementation 21: The implantable system of implementation 1, wherein the substrate comprises a first side and a second side opposite the first side, and wherein the plurality of conductive traces is present on the first and second sides of the substrate.

Implementation 22: The implantable system of implementation 21, wherein conductive traces present on the first side of substrate are connected to conductive traces present on the second side of the substrate with a conductive via.

Implementation 23: The implantable system of implementation 22, wherein the conductive via is made from one or more biocompatible materials.

Implementation 24: The implantable system of any of implementations 1 to 23, wherein the substrate comprises first and second substrates, and wherein the plurality of conductive traces is present on the first and second substrates.

Implementation 25: The implantable system of any of implementations 1 to 24, wherein the plurality of conductive traces is made from one or more biocompatible materials.

Implementation 26: The implantable system of any of implementations 1 to 25, wherein the antenna comprises a feed connecting the plurality of conductive traces to the communication circuitry.

Implementation 27: The implantable system of implementation 26, wherein the communication circuitry comprises a transceiver.

Implementation 28: The implantable system of any of implementations 26 to 27, wherein the feed connects the plurality of conductive traces to a ground of the electronic circuitry.

Implementation 29: The implantable system of implementation 28, wherein the ground comprises a floating ground plane.

Implementation 30: The implantable system of implementation 29, further comprising a housing made of conductive biocompatible material, the housing configured to support the electronic circuitry and the antenna, wherein the floating ground plane is connected to the housing.

Implementation 31: The implantable system of implementation 30, wherein the housing is made at least partially of titanium.

Implementation 32: The implantable system of any of implementations 1 to 31, wherein the plurality of conductive traces is present on a first side of the substrate, and wherein a ground plane is present on a second side of the substrate opposite the first side.

Implementation 33: The implantable system of implementation 32, wherein the plurality of conductive traces is connected to the ground plane via a feed or a via.

Implementation 34: The implantable system of any of implementations 1 to 33, wherein the substrate comprises first and second substrates, and wherein a first plurality of conductive traces is present on the first substrate and a second plurality of conductive traces is present on the second substrate.

Implementation 35: The implantable system of implementation 34, wherein the first plurality of conductive traces is connected to the second plurality of conductive traces with a conductive via.

Implementation 36: The implantable system of any of implementations 1 to 35, wherein the plurality of conductive traces is arranged in a zig-zag pattern.

Implementation 37: The implantable system of any of implementations 1 to 36, wherein the antenna comprises a planar inverted-F antenna.

Implementation 38: The implantable system of any of implementations 1 to 37, wherein the antenna is configured to transmit and receive in first and second distinct frequency bands.

Implementation 39: The implantable system of implementation 38, wherein the antenna resonates at center frequencies of the first and second frequency bands.

Implementation 40: The implantable system of any of implementations 38 to 39, wherein the first frequency band comprises medical device radiocommunications service (MICS) band and the second frequency band comprises industrial, scientific, and medical (ISM) band.

Implementation 41: The implantable system of any of implementations 38 to 40, wherein the electronic circuitry is configured to transition from a first power state to a second power state in which more power is consumed responsive to the antenna receiving a command in the second frequency band.

Implementation 42: The implantable system of implementation 41, wherein the first power state comprises a sleep state and the second power state comprises an operational state in which the processor is configured to cause transmission of the data by the communication circuitry and the antenna.

Implementation 43: The implantable system of implementation 42, wherein the data is transmitted in the first frequency band.

Implementation 44: The implantable system of any of implementations 42 to 43, wherein the processor is configured to cause transmission of the data in the second power state and not in the first power state.

Implementation 45: The implantable system of any of implementations 1 to 44, wherein the body part comprises a joint of the patient, and wherein at least one sensor is configured to monitor range of motion of the joint of the patient.

Implementation 46: The implantable system of implementation 45, wherein the at least one sensor comprises at least one or an accelerometer or gyroscope.

Implementation 47: The implantable system of any of implementations 1 to 46, wherein the body part comprises a hip joint, a knee joint, a shoulder joint, an elbow joint, or a spine.

Implementation 48: The implantable system of any of implementations 1 to 47, further comprising a femoral implant supporting the electronic circuitry, wherein the body part comprises a hip joint.

Implementation 49: The implantable system of any of implementations 1 to 48, wherein the substrate is made out of biocompatible material, such as one or more of liquid crystal polymer, polyimide, or polyamide.

Implementation 50: A kit comprising the implantable system of any of implementations 1 to 49 and a receiver configured to communicate with the communication circuitry.

Implementation 51: An implantable system for a spine of a patient, the system comprising:
an electronic circuitry comprising:
at least one sensor;
a processor configured to receive data obtained by the at least one sensor; and
a communication circuitry connected to the processor and configured to transmit the data obtained by the at least one sensor; and
an antenna comprising a plurality of conductive traces present on a substrate, the antenna connected to the communication circuitry and further configured to facilitate transmission of the data.

Implementation 52: The implantable system of implementation 51, wherein the plurality of conductive traces are painted or printed on the substrate.

Implementation 53: The implantable system of any of implementations 50 to 52, further comprising an interbody spacer or a spinal cage supporting the electronic circuitry. Implementation 54: The implantable system of any of implementations 51 to

53, wherein the plurality of conductive traces comprises a first plurality of conductive traces surrounding a second plurality of conductive traces, the first plurality of conductive traces connected to the second plurality of conductive traces.

Implementation 55: The implantable system of implementation 54, wherein first plurality of conductive traces is arranged in a first ring and the second plurality of conductive traces is arranged in a second ring, and wherein the first ring surrounds the second ring.

Implementation 56: The implantable system of implementation 55, wherein the substrate is circular.

Implementation 57: The implantable system of any of implementations 54 to 56, wherein the first plurality of conductive traces comprises a first plurality of interconnected petals and the second plurality of conductive traces comprises a second plurality of interconnected petals.

Implementation 58: The implantable system of implementation 57, wherein distance between petals in at least one of the first or second plurality of interconnected petals affects resonance of the antenna.

Implementation 59: The implantable system of any of implementations 57 to 58, wherein at least some petals in at least one of the first or second plurality of interconnected petals have rounded corners, thereby improving resonance of the antenna in one or more frequency bands of interest.

Implementation 60: The implantable system of any of implementations 57 to 59, wherein the plurality of conductive traces further comprises a stub connected to the second plurality of conductive traces.

Implementation 61: The implantable system of implementation 60, wherein the stub is curved.

Implementation 62: The implantable system of any of implementations 60 to 61, wherein the antenna is configured to transmit and receive in a frequency band, and wherein a length of the stub is selected to facilitate transmission and reception in the frequency band.

Implementation 63: The implantable system of any of implementations 51 to 62, wherein the substrate is supported by a spacer separating the antenna from the electronic circuitry.

Implementation 64: The implantable system of implementation 63, wherein the antenna further comprises an additional conductive trace supported by the spacer and connected to a conductive trace of the plurality of conductive traces.

Implementation 65: The implantable system of implementation 64, wherein the additional conductive trace is at least partially wrapped around the spacer.

Implementation 66: The implantable system of implementation 65, wherein the additional conductive trace is arranged into two sections that at least partially wrapped around opposite sides of the spacer.

Implementation 67: The implantable system of any of implementations 63 to 66, further comprising a cover enclosing the antenna and the spacer.

Implementation 68: The implantable system of implementation 67, wherein the cover is spherical, cylindrical, or spherical with a flat top.

Implementation 69: The implantable system of any of implementations 67 to 68, wherein the cover is made out of biocompatible material.

Implementation 70: The implantable system of any of implementations 51 to 69, wherein the substrate is made out of biocompatible material, such as one or more of liquid crystal polymer, polyimide, or polyamide.

Implementation 71: A kit comprising the implantable system of any of implementations 51 to 70 and a receiver configured to communicate with the communication circuitry.

Implementation 72: An implantable system for a body part of a patient, the system comprising:
an electronic circuitry comprising:
at least one sensor;
a processor configured to receive data obtained by the at least one sensor; and
a communication circuitry connected to the processor and configured to transmit the data obtained by the at least one sensor;
an antenna comprising a plurality of conductive traces present on a substrate, the antenna connected to the communication circuitry and further configured to facilitate transmission of the data;
a spacer supporting the substrate; and
an additional conductive trace supported by the spacer and connected to a conductive trace of the plurality of conductive traces.

Implementation 73: The implantable system of implementation 71, wherein the additional conductive trace is at least partially wrapped around the spacer.

Implementation 74: The implantable system of implementation 73, wherein the additional conductive trace is arranged into two sections that at least partially wrapped around opposite sides of the spacer.

Implementation 75: The implantable system of any of implementations 72 to 74, wherein the antenna is configured to transmit and receive in a frequency band, and wherein a height of the spacer is selected to facilitate transmission and reception in the frequency band.

Implementation 76: The implantable system of any of implementations 72 to 75, further comprising a cover enclosing the antenna and the spacer.

Implementation 77: The implantable system of implementation 76, wherein the cover is spherical, cylindrical, or spherical with a flat top.

Implementation 78: The implantable system of any of implementations 76 to 77, wherein the cover is made out of biocompatible material.

Implementation 79: The implantable system of any of implementations 72 to 78, wherein the spacer separates the antenna from the electronic circuitry.

Implementation 80: The implantable system of any of implementations 72 to 79, wherein the plurality of conductive traces are painted or printed on the substrate.

Implementation 81: The implantable system of any of implementations 72 to 80, wherein the spacer is made out of biocompatible material.

Implementation 82: The implantable system of any of implementations 72 to 81, wherein the plurality of conductive traces comprises a first plurality of conductive traces surrounding a second plurality of conductive traces, the first plurality of conductive traces connected to the second plurality of conductive traces.

Implementation 83: The implantable system of implementation 82, wherein the first plurality of conductive traces is arranged in a first ring and the second plurality of conductive traces is arranged in a second ring, and wherein the first ring surrounds the second ring.

Implementation 84: The implantable system of implementation 83, wherein the substrate is circular.

Implementation 85: The implantable system of any of implementations 82 to 84, wherein the first plurality of conductive traces comprises a first plurality of interconnected petals and the second plurality of conductive traces comprises a second plurality of interconnected petals.

Implementation 86: The implantable system of implementation 85, wherein distance between petals in at least one of the first or second plurality of interconnected petals affects resonance of the antenna.

Implementation 87: The implantable system of any of implementations 85 to 86, wherein at least some petals in at least one of the first or second plurality of interconnected petals have rounded corners, thereby improving resonance of the antenna in one or more frequency bands of interest.

Implementation 88: The implantable system of any of implementations 85 to 57, wherein the plurality of conductive traces further comprises a stub connected to the second plurality of conductive traces.

Implementation 89: The implantable system of implementation 88, wherein the stub is curved.

Implementation 90: The implantable system of any of implementations 88 to 89, wherein the antenna is configured to transmit and receive in a frequency band, and wherein a length of the stub is selected to facilitate transmission and reception in the frequency band.

Implementation 91: The implantable system of any of implementations 72 to 90, wherein the substrate is made out of biocompatible material, such as one or more of liquid crystal polymer, polyimide, or polyamide.

Implementation 92: The implantable system of any of implementations 72 to 91, wherein the body part comprises a hip joint, a knee joint, a shoulder joint, an elbow joint, or a spine.

Implementation 93: The implantable system of any of implementations 72 to 92, further comprising a femoral implant supporting the electronic circuitry, wherein the body part comprises a hip joint.

Implementation 94: An antenna for use in an implantable system for a body part of a patient, the antenna comprising:
a substrate; and
a plurality of conductive traces present on the substrate, the antenna configured to be connected to a communication circuitry and facilitate transmission of data.

Implementation 95: The antenna of implementation 94, further comprising an additional conductive trace positioned on a spacer that supports the substrate, the additional conductive trace connected to a conductive trace of the plurality of conductive traces.

Implementation 96: The antenna of implementation 95, wherein the additional conductive trace is at least partially wrapped around the spacer.

Implementation 97: The antenna of implementation 96, wherein the additional conductive trace is arranged into two sections that at least partially wrapped around opposite sides of the spacer.

Implementation 98: The antenna of any of implementations 95 to 97, wherein the antenna is configured to transmit and receive in a frequency band, and wherein a height of the spacer is selected to facilitate transmission and reception in the frequency band.

Implementation 99: The antenna of any of implementations 95 to 98, further comprising a cover enclosing the antenna and the spacer.

Implementation 100: The antenna of implementation 99, wherein the cover is spherical, cylindrical, or spherical with a flat top.

Implementation 101: The antenna of any of implementations 99 to 100, wherein the cover is made out of biocompatible material.

Implementation 102: The antenna of any of implementations 95 to 101, wherein the spacer is configured to separate the antenna from the communication circuitry.

Implementation 103: The antenna of any of implementations 94 to 102, wherein the plurality of conductive traces are painted or printed on the substrate.

Implementation 104: The antenna of any of implementations 94 to 103, wherein the substrate comprises biocompatible material.

Implementation 105: The antenna of implementation 104, wherein the biocompatible material comprises at least one of liquid crystal polymer, polyimide, or polyamide.

Implementation 106: The antenna of any of implementations 94 to 105, wherein the plurality of conductive traces comprises a first plurality of conductive traces surrounding a second plurality of conductive traces, the first plurality of conductive traces connected to the second plurality of conductive traces.

Implementation 107: The antenna of implementation 106, wherein the first plurality of conductive traces is arranged in a first ring and the second plurality of conductive traces is arranged in a second ring, and wherein the first ring surrounds the second ring.

Implementation 108: The antenna of implementation 107, wherein the substrate is circular.

Implementation 109: The antenna of any of implementation 106 to 108, wherein the first plurality of conductive traces comprises a first plurality of interconnected petals and the second plurality of conductive traces comprises a second plurality of interconnected petals.

Implementation 110: The antenna of implementation 109, wherein distance between petals in at least one of the first or second plurality of interconnected petals affects resonance of the antenna.

Implementation 111: The antenna of implementation 110, wherein at least some petals in at least one of the first or second plurality of interconnected petals have rounded corners, thereby improving resonance of the antenna in one or more frequency bands of interest.

Implementation 112: The antenna of any of implementations 110 to 111, wherein the plurality of conductive traces further comprises a stub connected to the second plurality of conductive traces.

Implementation 113: The antenna of implementation 112, wherein the stub is curved.

Implementation 114: The antenna of any of implementations 112 to 113, wherein the antenna is configured to transmit and receive in a frequency band, and wherein a length of the stub is selected to facilitate transmission and reception in the frequency band.

What is claimed is:

1. An implantable system for a body part of a patient, the system comprising:
an electronic circuitry comprising:
at least one sensor;
a processor configured to receive data obtained by the at least one sensor; and
a communication circuitry connected to the processor and configured to transmit the data obtained by the at least one sensor; and
an antenna comprising a plurality of conductive traces present on a biocompatible substrate, the antenna connected to the communication circuitry and further configured to facilitate transmission of the data.

2. The implantable system of claim 1, wherein the plurality of conductive traces are painted or printed on the biocompatible substrate.

3. The implantable system of claim 1, wherein the biocompatible substrate comprises at least one of liquid crystal polymer, polyimide, or polyamide.

4. The implantable system of claim 1, wherein the plurality of conductive traces comprises a first plurality of conductive traces surrounding a second plurality of conductive traces, the first plurality of conductive traces connected to the second plurality of conductive traces.

5. The implantable system of claim 4, wherein the first plurality of conductive traces is arranged in a first ring and the second plurality of conductive traces is arranged in a second ring, and wherein the first ring surrounds the second ring.

6. The implantable system of claim 5, wherein the biocompatible substrate is circular.

7. The implantable system of claim 4, wherein the first plurality of conductive traces comprises a first plurality of interconnected petals and the second plurality of conductive traces comprises a second plurality of interconnected petals.

8. The implantable system of claim 7, wherein distance between petals in at least one of the first or second plurality of interconnected petals affects resonance of the antenna.

9. The implantable system of claim 7, wherein at least some petals in at least one of the first or second plurality of interconnected petals have rounded corners, thereby improving resonance of the antenna in one or more frequency bands of interest.

10. The implantable system of claim 7, wherein the plurality of conductive traces further comprises a stub connected to the second plurality of conductive traces.

11. The implantable system of claim 10, wherein the stub is curved.

12. The implantable system of claim 10, wherein the antenna is configured to transmit and receive in a frequency band, and wherein a length of the stub is selected to facilitate transmission and reception in the frequency band.

13. The implantable system of claim 1, wherein the biocompatible substrate is supported by a spacer separating the antenna from the electronic circuitry.

14. The implantable system of claim 13, wherein the antenna further comprises an additional conductive trace supported by the spacer and connected to a conductive trace of the plurality of conductive traces.

15. The implantable system of claim 14, wherein the additional conductive trace is at least partially wrapped around the spacer.

16. The implantable system of claim 15, wherein the additional conductive trace is arranged into two sections that at least partially wrapped around opposite sides of the spacer.

17. The implantable system of claim 13, wherein the antenna is configured to transmit and receive in a frequency band, and wherein a height of the spacer is selected to facilitate transmission and reception in the frequency band.

18. The implantable system of claim 13, further comprising a cover enclosing the antenna and the spacer.

19. The implantable system of claim 18, wherein the cover is spherical, cylindrical, or spherical with a flat top.

20. The implantable system of claim 18, wherein the cover is made out of biocompatible material.

21. The implantable system of claim 1, wherein the biocompatible substrate comprises a first side and a second side opposite the first side, and wherein the plurality of conductive traces is present on the first and second sides of the biocompatible substrate.

22. The implantable system of claim 21, wherein conductive traces present on the first side of substrate are connected to conductive traces present on the second side of the substrate with a conductive via.

23. The implantable system of claim 22, wherein the conductive via is made from one or more biocompatible materials.

24. The implantable system of claim 1, wherein the biocompatible substrate comprises a first and second biocompatible substrates, and wherein the plurality of conductive traces is present on the first and second biocompatible substrates.

25. The implantable system of claim 1, wherein the plurality of conductive traces is made from one or more biocompatible materials.

26. The implantable system of claim 1, wherein the antenna comprises a feed connecting the plurality of conductive traces to the communication circuitry.

27. The implantable system of claim 26, wherein the communication circuitry comprises a transceiver.

28. The implantable system of claim 26, wherein the feed connects the plurality of conductive traces to a ground of the electronic circuitry.

29. The implantable system of claim 28, wherein the ground comprises a floating ground plane.

30. The implantable system of claim 29, further comprising a housing made of conductive biocompatible material, the housing configured to support the electronic circuitry and the antenna, wherein the floating ground plane is connected to the housing.

31. The implantable system of claim 30, wherein the housing is made at least partially of titanium.

32. The implantable system of claim 1, wherein the plurality of conductive traces is present on a first side of the biocompatible substrate, and wherein a ground plane is present on a second side of the biocompatible substrate opposite the first side.

33. The implantable system of claim 32, wherein the plurality of conductive traces is connected to the ground plane via a feed or a via.

34. The implantable system of claim 1, wherein the biocompatible substrate comprises first and second biocompatible substrates, and wherein a first plurality of conductive traces is present on the first biocompatible substrate and a second plurality of conductive traces is present on the second biocompatible substrate.

35. The implantable system of claim 34, wherein the first plurality of conductive traces is connected to the second plurality of conductive traces with a conductive via.

36. The implantable system of claim 1, wherein the plurality of conductive traces is arranged in a zig-zag pattern.

37. The implantable system of claim 1, wherein the antenna comprises a planar inverted-F antenna.

38. The implantable system of claim 1, wherein the antenna is configured to transmit and receive in first and second distinct frequency bands.

39. The implantable system of claim 38, wherein the antenna resonates at center frequencies of the first and second frequency bands.

40. The implantable system of claim 38, wherein the first frequency band comprises medical device radiocommunications service (MICS) band and the second frequency band comprises industrial, scientific, and medical (ISM) band.

41. The implantable system of claim 38, wherein the electronic circuitry is configured to transition from a first power state to a second power state in which more power is consumed responsive to the antenna receiving a command in the second frequency band.

42. The implantable system of claim 41, wherein the first power state comprises a sleep state and the second power state comprises an operational state in which the processor is configured to cause transmission of the data by the communication circuitry and the antenna.

43. The implantable system of claim 42, wherein the data is transmitted in the first frequency band.

44. The implantable system of claim 42, wherein the processor is configured to cause transmission of the data in the second power state and not in the first power state.

45. The implantable system of claim 1, wherein the body part comprises a joint of the patient, and wherein at least one sensor is configured to monitor range of motion of the joint of the patient.

46. The implantable system of claim 45, wherein the at least one sensor comprises at least one or an accelerometer or gyroscope.

47. The implantable system of claim 1, wherein the body part comprises a hip joint, a knee joint, a shoulder joint, an elbow joint, or a spine.

48. The implantable system of claim 1, further comprising a femoral implant supporting the electronic circuitry, wherein the body part comprises a hip joint.

49. A kit comprising the implantable system of claim 1 and a receiver configured to communicate with the communication circuitry.

50. An implantable system for a spine of a patient, the system comprising:
an electronic circuitry comprising:
at least one sensor;
a processor configured to receive data obtained by the at least one sensor; and
a communication circuitry connected to the processor and configured to transmit the data obtained by the at least one sensor; and
an antenna comprising a plurality of conductive traces present on a biocompatible substrate, the antenna connected to the communication circuitry and further configured to facilitate transmission of the data.

51. The implantable system of claim 50, wherein the plurality of conductive traces are painted or printed on the biocompatible substrate.

52. The implantable system of claim 50, further comprising an interbody spacer or a spinal cage supporting the electronic circuitry.

53. The implantable system of claim 50, wherein the plurality of conductive traces comprises a first plurality of conductive traces surrounding a second plurality of conductive traces, the first plurality of conductive traces connected to the second plurality of conductive traces.

54. The implantable system of claim 53, wherein first plurality of conductive traces is arranged in a first ring and the second plurality of conductive traces is arranged in a second ring, and wherein the first ring surrounds the second ring.

55. The implantable system of claim 54, wherein the biocompatible substrate is circular.

56. The implantable system of claim 53, wherein the first plurality of conductive traces comprises a first plurality of interconnected petals and the second plurality of conductive traces comprises a second plurality of interconnected petals.

57. The implantable system of claim 56, wherein distance between petals in at least one of the first or second plurality of interconnected petals affects resonance of the antenna.

58. The implantable system of claim 56, wherein at least some petals in at least one of the first or second plurality of interconnected petals have rounded corners, thereby improving resonance of the antenna in one or more frequency bands of interest.

59. The implantable system of claim 56, wherein the plurality of conductive traces further comprises a stub connected to the second plurality of conductive traces.

60. The implantable system of claim 59, wherein the stub is curved.

61. The implantable system of claim 59, wherein the antenna is configured to transmit and receive in a frequency band, and wherein a length of the stub is selected to facilitate transmission and reception in the frequency band.

62. The implantable system of claim 50, wherein the biocompatible substrate is supported by a spacer separating the antenna from the electronic circuitry.

63. The implantable system of claim 62, wherein the antenna further comprises an additional conductive trace supported by the spacer and connected to a conductive trace of the plurality of conductive traces.

64. The implantable system of claim 63, wherein the additional conductive trace is at least partially wrapped around the spacer.

65. The implantable system of claim 64, wherein the additional conductive trace is arranged into two sections that at least partially wrapped around opposite sides of the spacer.

66. The implantable system of claim 62, further comprising a cover enclosing the antenna and the spacer.

67. The implantable system of claim 66, wherein the cover is spherical, cylindrical, or spherical with a flat top.

68. The implantable system of claim 66, wherein the cover is made out of biocompatible material.

69. A kit comprising the implantable system of claim 50 and a receiver configured to communicate with the communication circuitry.

70. An implantable system for a body part of a patient, the system comprising:
an electronic circuitry comprising:
at least one sensor;
a processor configured to receive data obtained by the at least one sensor; and
a communication circuitry connected to the processor and configured to transmit the data obtained by the at least one sensor;
an antenna comprising a plurality of conductive traces present on a biocompatible substrate, the antenna connected to the communication circuitry and further configured to facilitate transmission of the data;
a spacer supporting the biocompatible substrate; and
an additional conductive trace supported by the spacer and connected to a conductive trace of the plurality of conductive traces.

71. The implantable system of claim 70, wherein the additional conductive trace is at least partially wrapped around the spacer.

72. The implantable system of claim 71, wherein the additional conductive trace is arranged into two sections that at least partially wrapped around opposite sides of the spacer.

73. The implantable system of claim 70, wherein the antenna is configured to transmit and receive in a frequency band, and wherein a height of the spacer is selected to facilitate transmission and reception in the frequency band.

74. The implantable system of claim 70, further comprising a cover enclosing the antenna and the spacer.

75. The implantable system of claim 74, wherein the cover is spherical, cylindrical, or spherical with a flat top.

76. The implantable system of claim 74, wherein the cover is made out of biocompatible material.

77. The implantable system of claim 70, wherein the spacer separates the antenna from the electronic circuitry.

78. The implantable system of claim 70, wherein the plurality of conductive traces are painted or printed on the biocompatible substrate.

79. The implantable system of claim 70, wherein the spacer is made out of biocompatible material.

80. The implantable system of claim 70, wherein the plurality of conductive traces comprises a first plurality of conductive traces surrounding a second plurality of conductive traces, the first plurality of conductive traces connected to the second plurality of conductive traces.

81. The implantable system of claim 80, wherein the first plurality of conductive traces is arranged in a first ring and the second plurality of conductive traces is arranged in a second ring, and wherein the first ring surrounds the second ring.

82. The implantable system of claim 81, wherein the biocompatible substrate is circular.

83. The implantable system of claim 80, wherein the first plurality of conductive traces comprises a first plurality of interconnected petals and the second plurality of conductive traces comprises a second plurality of interconnected petals.

84. The implantable system of claim 83, wherein distance between petals in at least one of the first or second plurality of interconnected petals affects resonance of the antenna.

85. The implantable system of claim 83, wherein at least some petals in at least one of the first or second plurality of interconnected petals have rounded corners, thereby improving resonance of the antenna in one or more frequency bands of interest.

86. The implantable system of claim 83, wherein the plurality of conductive traces further comprises a stub connected to the second plurality of conductive traces.

87. The implantable system of claim 86, wherein the stub is curved.

88. The implantable system of claim 86, wherein the antenna is configured to transmit and receive in a frequency band, and wherein a length of the stub is selected to facilitate transmission and reception in the frequency band.

89. The implantable system of claim 70, wherein the biocompatible substrate comprises at least one of liquid crystal polymer, polyimide, or polyamide.

90. The implantable system of claim 70, wherein the body part comprises a hip joint, a knee joint, a shoulder joint, an elbow joint, or a spine.

91. The implantable system of claim 70, further comprising a femoral implant supporting the electronic circuitry, wherein the body part comprises a hip joint.

92. An antenna for use in an implantable system for a body part of a patient, the antenna comprising:
a substrate; and
a plurality of conductive traces present on the substrate, the antenna configured to be connected to a communication circuitry and facilitate transmission of data.

93. The antenna of claim 92, further comprising an additional conductive trace positioned on a spacer that supports the substrate, the additional conductive trace connected to a conductive trace of the plurality of conductive traces.

94. The antenna of claim 93, wherein the additional conductive trace is at least partially wrapped around the spacer.

95. The antenna of claim 94, wherein the additional conductive trace is arranged into two sections that at least partially wrapped around opposite sides of the spacer.

96. The antenna of claim 93, wherein the antenna is configured to transmit and receive in a frequency band, and wherein a height of the spacer is selected to facilitate transmission and reception in the frequency band.

97. The antenna of claim 93, further comprising a cover enclosing the antenna and the spacer.

98. The antenna of claim 97, wherein the cover is spherical, cylindrical, or spherical with a flat top.

99. The antenna of claim 97, wherein the cover is made out of biocompatible material.

100. The antenna of claim 93, wherein the spacer is configured to separate the antenna from the communication circuitry.

101. The antenna of claim 92, wherein the plurality of conductive traces are painted or printed on the substrate.

102. The antenna of claim 92, wherein the substrate comprises biocompatible material.

103. The antenna of claim 102, wherein the biocompatible material comprises at least one of liquid crystal polymer, polyimide, or polyamide.

104. The antenna of claim 92, wherein the plurality of conductive traces comprises a first plurality of conductive traces surrounding a second plurality of conductive traces, the first plurality of conductive traces connected to the second plurality of conductive traces.

105. The antenna of claim 104, wherein the first plurality of conductive traces is arranged in a first ring and the second plurality of conductive traces is arranged in a second ring, and wherein the first ring surrounds the second ring.

106. The antenna of claim 105, wherein the substrate is circular.

107. The antenna of claim 104, wherein the first plurality of conductive traces comprises a first plurality of interconnected petals and the second plurality of conductive traces comprises a second plurality of interconnected petals.

108. The antenna of claim 107, wherein distance between petals in at least one of the first or second plurality of interconnected petals affects resonance of the antenna.

109. The antenna of claim 108, wherein at least some petals in at least one of the first or second plurality of interconnected petals have rounded corners, thereby improving resonance of the antenna in one or more frequency bands of interest.

110. The antenna of claim 108, wherein the plurality of conductive traces further comprises a stub connected to the second plurality of conductive traces.

111. The antenna of claim 110, wherein the stub is curved.

112. The antenna of claim 110, wherein the antenna is configured to transmit and receive in a frequency band, and wherein a length of the stub is selected to facilitate transmission and reception in the frequency band.
